(19) United States
(12) Patent Application Publication
(10) Pub. No.: US 2024/0145034 A1
(43) Pub. Date: May 2, 2024

(54) METHODS OF IDENTIFYING A CONDENSATE PHENOTYPE AND USES THEREOF

(71) Applicant: Dewpoint Therapeutics, Inc., Boston, MA (US)

(72) Inventors: William W. CHEN, Winchester, MA (US); John C. MANTEIGA, Boston, MA (US); Violeta YU, Wakefield, MA (US); Ann D. KWONG, Pepperell, MA (US); Peter Jeffrey DANDLIKER, Quincy, MA (US); Bruce Aaron BEUTEL, Needham, MA (US); Chi ZHANG, Westford, MA (US); Lingyao ZENG, Wuppertal (DE); Andreas STEFFEN, Berlin (DE); Daniel Franz FREITAG, Duesseldorf (DE)

(21) Appl. No.: 18/279,760

(22) PCT Filed: Mar. 1, 2022
(86) PCT No.: PCT/US2022/018311

§ 371 Date: Aug. 31, 2023

Related U.S. Application Data
(60) Provisional application No. 63/298,171, filed on Jan. 10, 2022.
(60) Provisional application No. 63/155,683, filed on Mar. 2, 2021.

(51) Int. Cl. G16B 20/20; C12N 15/10; G16B 40/20; G16H 70/40

(52) U.S. Cl. CPC G16B 20/20; C12N 15/1079; C12N 15/1089; G16B 40/20; G16H 70/40

(57) ABSTRACT

The present application provides, in some aspects, methods of identifying a condensate of interest associated with a el disease. Also provided are methods of identifying a marker for identifying a condensate of interest associated with a disease. In other aspects, provided herein are methods of identifying a therapeutic agent useful for treating a disease via the identified condensate of interest.

### CROSS REFERENCE TO RELATED APPLICATIONS

This application claims priority benefit of U.S. Provisional Patent Application No. 63/155,683, filed on Mar. 2, 2021, and U.S. Provisional Patent Application No. 63/298,171, filed on Jan. 10, 2022, the content of each of which is incorporated herein by reference in their entireties.

### TECHNICAL FIELD

The present application relates to the field of biological condensates.

### BACKGROUND

Conventional disease research and application to therapeutic drug discovery focuses on identifying individual biomolecules (molecular targets) that cause or mediate disease-relevant biology, such as via known cellular pathways. Two principles are integral with this therapeutic drug discovery approach: (1) the molecular target needs to be validated (e.g., using genetics or pharmacology), to provide evidence that modulation of intrinsic molecular target activity and/or interactions with other molecules may act to alleviate disease biology; and, (2) the molecular target needs to be “druggable,” with at least one therapeutic modality (e.g., a small molecule or antibody) capable of effecting the desired impact on the molecular target modulation in vivo. This “single target” approach to disease research and therapeutic drug discovery has significant limitations as in vivo systems are extraordinarily complex and our understanding of cellular pathways and biomolecular actions and interactions are often incomplete; moreover, not all individual biomolecules, when viewed singularly, are suitable drug targets. Such challenges have contributed to the current shortage of new therapeutic drugs to treat unmet medical needs.

### BRIEF SUMMARY

In some aspects, provided herein is a method of identifying a condensate of interest associated with a disease, the method comprising: (a) comparing a first condensate phenotype from a first cell model with a second condensate phenotype from a second cell model, wherein a difference between the first cell model and the second cell model is attributable to one or more disease-associated factors, and wherein at least one of the one or more disease-associated factors is introduced to either the first cell model or the second cell model; and (b) identifying the condensate of interest as associated with the disease based on a difference between the first condensate phenotype and the second condensate phenotype for the condensate of interest.

In some embodiments according to any one of the methods described herein, the first condensate phenotype and the second condensate phenotype are each characterized by one or more phenotypic identifiers. In some embodiments, the one or more phenotypic identifiers comprise an identifier selected from the group consisting of a condensate presence, absence, level, morphological feature, location, behavior, composition, and material property.

In some embodiments according to any one of the methods described herein, the one or more disease-associated factors associated with the disease comprise a factor selected from the group consisting of a genetic variant, post-translational modification variant, exogenous genetic material, presence of an endogenous compound, presence of an exogenous compound, a physical process (e.g., stress), and environmental stimulus.

In some embodiments according to any one of the methods described herein, the second cell model is treated and/or engineered based on the one or more disease-associated factors associated with the disease.

In some embodiments according to any one of the methods described herein, the first cell model is treated and/or engineered based on the one or more disease-associated factors associated with the disease.

In some embodiments according to any one of the methods described herein, the method further comprises obtaining the second cell model.

In some embodiments according to any one of the methods described herein, the method further comprises producing the second cell model.

In some embodiments according to any one of the methods described herein, the method further comprises obtaining the first cell model.

In some embodiments according to any one of the methods described herein, the method further comprises producing the first cell model.

In some embodiments according to any one of the methods described herein, the method further comprises obtaining the first condensate phenotype. In some embodiments, obtaining the first condensate phenotype comprises measuring an association of a first marker with the condensate of interest. In some embodiments, the first marker is a biological marker. In some embodiments, the association of the first marker with the condensate of interest is determined using an imaging technique. In some embodiments, the imaging technique comprises labeling the first marker.

In some embodiments according to any one of the methods described herein, the method further comprises obtaining the second condensate phenotype. In some embodiments, obtaining the second condensate phenotype comprises measuring an association of a second marker with the condensate of interest. In some embodiments, the second marker is a biological marker. In some embodiments, the association of the second marker with the condensate of interest is determined using an imaging technique. In some embodiments, the imaging technique comprises labeling the second marker.

In some embodiments according to any one of the methods described herein, the method further comprises determining the difference between the first condensate phenotype and the second condensate phenotype. In some embodiments, determining the difference between the first condensate phenotype and the second condensate phenotype comprises a qualitative assessment. In some embodiments, determining the difference between the first condensate phenotype and the second condensate phenotype comprises a quantitative assessment. In some embodiments, determining the difference between the first condensate phenotype and the second condensate phenotype comprises an in silico technique.

In some embodiments according to any one of the methods described herein, the condensate of interest is present in, or derived from, the first cell model.

In some embodiments according to any one of the methods described herein, the condensate of interest is absent in, or not derived from, the first cell model.

In some embodiments according to any one of the methods described herein, the condensate of interest is absent in, or not derived from, the second cell model.

In some embodiments according to any one of the methods described herein, the condensate of interest is present in, or derived from, the second cell model.

In some embodiments according to any one of the methods described herein, the condensate of interest belongs to a condensate type selected from the group consisting of a stress granule, cleavage body, p-granule, histone locus body, multivesicular body, neuronal RNA granule, nuclear gem, nuclear pore, nuclear speckle, nuclear stress body, nucleolus, Oct1/PTF/transcription (OPT) domain, paraspeckle, perinucleolar compartment, PML, nuclear body, PML, oncogenic domain, polycomb body, processing body, Sam68 nuclear body, and splicing speckle.

In some embodiments according to any one of the methods described herein, the disease is a monogenic disease.

In some embodiments according to any one of the methods described herein, the disease is a polygenic disease.

In some embodiments according to any one of the methods described herein, the disease is a multifactorial disease.

In some embodiments according to any one of the methods described herein, the disease is caused, at least in part, by a stimulus and/or an exogenous agent.

In some embodiments according to any one of the methods described herein, the disease is caused by an infectious agent.

In some embodiments according to any one of the methods described herein, the first marker and the second marker are the same. In some embodiments, the first marker and the second marker are different.

In other aspects, provided herein is a method of identifying a marker useful for identifying a condensate of interest associated with a disease, the method comprising: (a) assessing a plurality of candidate markers, or precursors thereof, for a level of association with one or more disease-associated factors of the disease; (b) assessing at least one of the plurality of candidate markers for a condensate affinity factor; and (c) identifying the marker from the plurality of candidate markers based on the marker having a desired level of association with the one or more disease-associated factors of the disease and having a desired condensate affinity factor.

In some embodiments according to any one of the methods described herein, the method further comprises identifying the one or more disease-associated factors of the disease. In some embodiments, each of the one or more disease-associated factors of the disease is selected from the group consisting of a genetic variant, post translational modification variant, exogenous genetic material, presence of an endogenous compound, presence of an exogenous compound, a physical process, and environmental stimulus.

In some embodiments according to any one of the methods described herein, the method further comprises identifying a gene or a non-coding variant associated with the one or more disease-associated factors of the disease.

In some embodiments according to any one of the methods described herein, the level of association of each candidate marker with the one or more disease-associated factors of the disease is based on a disease-causal factor score. In some embodiments, the disease-causal factor score reflects the strength of association of each candidate marker with the one or more disease-associated factors of the disease. In some embodiments, the method further comprises assigning each candidate marker with the disease-causal factor score.

In some embodiments according to any one of the methods described herein, the condensate affinity factor is based on a condensate-association score. In some embodiments, the condensate-association score reflects the strength of association of the candidate marker, or a portion thereof, with any condensate, a specific condensate, and/or a macromolecule associated with a condensate. In some embodiments, the method further comprises assigning the candidate marker with the condensate-association score. In some embodiments, the condensate-association score is a composite score of a condensate function score and a condensate affinity score. In some embodiments, the condensate function score is determined based on one or more factors of whether a genetic variation of the candidate marker or a portion thereof or the gene or the non-coding variant associated with the one or more disease-associated factors of the disease: i) is within an intrinsically disordered region (IDR); ii) is subject to a post-translational modification; iii) affects splicing of the candidate marker or the gene associated with the one or more disease-associated factors of the disease; iv) affects a chromatin state close to the gene or the non-coding variant associated with the one or more disease-associated factors of the disease; and v) affects expression of the gene associated with the one or more disease-associated factors of the disease. In some embodiments, the one or more factors for determining the condensate function score each has a weight contributing to the condensate function score. In some embodiments, the condensate affinity score is determined based on, in the candidate marker or a portion thereof or the gene associated with the one or more disease-associated factors of the disease, one or more factors of: i) the presence, absence, amount, and/or degree of an IDR; ii) the presence, absence, amount, and/or degree of a condensate-favoring motif; and iii) the presence, absence, amount, and/or valency of an interacting domain. In some embodiments, the one or more factors for determining the condensate affinity score each has a weight contributing to the condensate affinity score.

In some embodiments according to any one of the methods described herein, the method further comprises identifying a gene expression product based on the identified gene, wherein the gene expression product, or a portion thereof, is used to populate the plurality of candidate markers, or precursors thereof.

In some embodiments according to any one of the methods described herein, identifying the marker from the plurality of candidate markers comprises a cumulative score based on the desired level of association with the one or more disease-associated factors of the disease and the desired condensate affinity factor.

In some embodiments according to any one of the methods described herein, the marker is a biological marker.

In some embodiments according to any one of the methods described herein, the marker is identified in silico.

In some embodiments according to any one of the methods described herein, the method further comprises verifying the marker as useful for identifying a condensate of interest associated with the disease.

In other aspects, provided herein is a method of identifying a condensate of interest associated with a disease, the method comprising: (a) comparing a first condensate phenotype from a first cell model with a second condensate phenotype from a second cell model, wherein the first condensate phenotype and the second condensate phenotype are obtained using a marker identified using a method described herein (e.g., any one of the methods described above), wherein a difference between the first cell model and the second cell model is attributable to one or more disease-associated factors of the disease, and wherein at least one of the one or more disease-associated factors is introduced to either the first cell model or the second cell model; and (b) identifying the condensate of interest as associated with the disease based on a difference between the first condensate phenotype and the second condensate phenotype for the condensate of interest.

In other aspects, provided herein is a method of identifying a compound that modulates a condensate phenotype, the method comprising: (a) admixing the compound and a composition comprising a cell model; and (b) obtaining a resulting condensate phenotype of the composition, wherein a difference between the resulting condensate phenotype and a reference condensate phenotype identifies the compound as modulating the condensate phenotype.

In other aspects, provided herein is a method of identifying a compound useful for treating a disease, the method comprising: (a) admixing the compound and a composition comprising a first cell model; (b) obtaining a resulting condensate phenotype of the composition, wherein the compound is identified as useful for treating the disease when the resulting condensate phenotype has a desired modulation of a phenotypic identifier associated with one or more disease-associated factors of the disease.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

All references cited herein, including patent applications and publications, are incorporated herein by reference in their entirety.

### BRIEF DESCRIPTION OF THE DRAWINGS

FIGS. 1A-1D show fluorescent images of H9C2 cells transiently transfected with a wild type RBM20 polypeptide (FIG. 1A), a R636S mutant RBM20 polypeptide (FIG. 1B), a R636C mutant RBM20 polypeptide (FIG. 1C), and a R636H mutant RBM20 polypeptide (FIG. 1D).

FIG. 2 shows a schematic of an exemplary workflow for evaluating genes within lead single nucleotide polymorphisms (SNPs).

FIG. 3 shows enrichment of SNPs across the genomic loci of KCNJ11 and ABCC8.

FIGS. 4A-4B show fluorescent images of iCell® Cardiomyocytes transiently transfected with GFP fused with a wild type Desmoplakin (DSP) polypeptide (FIG. 4A), a S299R mutant DSP polypeptide (FIG. 4B), and a Q331ter termination mutant DSP polypeptide (FIG. 4C). Cell DNA was also stained for DAPI.

FIGS. 5A-5B show fluorescent images of iCell® Cardiomyocytes transiently transfected with GFP fused with a wild type Desmoglein-2 (DSG2) polypeptide (FIG. 5A), and a W306ter termination mutant DSG2 polypeptide (FIG. 5B). Cell DNA was also stained with DAPI.

FIGS. 6A-6F show fluorescent images of iCell® Cardiomyocytes transiently transfected with GFP fused with a wild type alpha-protein kinase 3 (ALPK3) polypeptide (FIG. 6A), an L1299P mutant ALPK3 polypeptide (FIG. 6B), an L1622P mutant ALPK3 polypeptide (FIG. 6C), an R1261ter termination mutant ALPK3 polypeptide (FIG. 6D), a W1264ter termination mutant ALPK3 polypeptide (FIG. 6E), and a W1765ter termination mutant ALPK3 polypeptide (FIG. 6F). Cell DNA was also stained with DAPI.

### DETAILED DESCRIPTION

The present application provides, in some aspects, methods of identifying a condensate phenotype associated with a disease. Condensates are membrane-less molecular assemblies formed through liquid-liquid phase separation. Condensates are highly dynamic hubs bringing together many molecules, including endogenous and exogenous molecules. In some embodiments, the condensate phenotype is characterized by one or more phenotypic identifiers such as the presence, absence, amount, morphological feature (e.g., shape, size, sphericity), location (e.g., cytoplasm vs. nucleus), distribution (e.g., relative to a cellular organelle), behavior (e.g., kinetics), composition, or material property (e.g., fluidity, fiber-like or gel-like) of a condensate. In some embodiments, the condensate phenotype may elucidate a disease mechanism (or portion thereof) and/or a point of therapeutic intervention useful for treating the disease. In some embodiments, the condensate phenotypes may elucidate a functional mechanism by which a factor (e.g., causal factor) of a disease (such as a condensate and/or a biological molecule) manifests into observed disease biology, and thus, in some instances, provide a therapeutic target. The present disclosure is based, at least in part, on the inventors' findings and unique perspectives regarding, e.g., the role of condensates in disease biology, the development of relevant cell models for studying condensates and condensate phenotypes, and the development of methods for the identification of disease-associated condensate phenotypes, identification of condensates of interest associated with diseases, and identification of therapeutic agents for treating a disease, such as by modulating a condensate phenotype and/or condensate of interest.

In some aspects, provided herein are methods of identifying a condensate phenotype associated with a disease. In some embodiments, the condensate phenotype is used to identify a condensate of interest associated with the disease. In some embodiments, the identified condensate phenotype and/or the condensate of interest associated with the disease are used to identify a factor (e.g., causal factor) associated with the disease. In other aspects, provided herein are methodologies for imaging condensate phenotypes and/or condensates of interest. In some embodiments, the imaging techniques utilize a marker, such as a biological marker, to observe, e.g., condensates, condensate components, and/or cellular features. In some embodiments, provided herein are markers (such as marker panels) and methods for identifying markers useful for the imaging techniques described herein. In other aspects, provided herein are cell models useful for the methods described herein. For example, in some embodiments, the comparison of two cell models enables the identification of a condensate phenotype and/or condensate of interest. In some embodiments, provided herein are methodologies for designing and engineering such cell models.

These findings and perspectives create new opportunities for drug development by identifying new druggable molecular targets (including, but not limited to, single molecular targets and communities of polypeptides and/or nucleic acids such as biological condensates) and methods for identifying compounds useful for treating diseases. For example, the methods described herein are useful for identifying and validating a condensate-based hypothesis for a multifactorial disease, including diseases influenced by polygenic and/or environmental factors.

Thus, in some aspects, provided herein is a method of identifying a condensate of interest associated with a disease, the method comprising: (a) comparing a first condensate phenotype from a first cell model with a second condensate phenotype from a second cell model, wherein a difference between the first cell model and the second cell model is attributable to one or more disease-associated factors, and wherein at least one of the one or more disease-associated factors is introduced to either the first cell model or the second cell model; and (b) identifying the condensate of interest as associated with the disease based on a difference between the first condensate phenotype and the second condensate phenotype for the condensate of interest.

In other aspects, provided herein is a method of identifying a condensate of interest associated with a disease, the method comprising: (a) comparing a first condensate phenotype from a first cell model with a second condensate phenotype from a second cell model, wherein the first condensate phenotype and the second condensate phenotype are obtained using a marker identified using a method described herein, wherein a difference between the first cell model and the second cell model is attributable to one or more disease-associated factors of the disease, and wherein at least one of the one or more disease-associated factors is introduced to either the first cell model or the second cell model; and (b) identifying the condensate of interest as associated with the disease based on a difference between the first condensate phenotype and the second condensate phenotype for the condensate of interest.

In other aspects, provided herein is a method of identifying a compound that modulates a condensate phenotype, the method comprising: (a) admixing the compound and a composition comprising a cell model; and (b) obtaining a resulting condensate phenotype of the composition, wherein a difference between the resulting condensate phenotype and a reference condensate phenotype identifies the compound as modulating the condensate phenotype.

In other aspects, provided herein is a method of identifying a compound useful for treating a disease, the method comprising: (a) admixing the compound and a composition comprising a first cell model; (b) obtaining a resulting condensate phenotype of the composition, wherein the compound is identified as useful for treating the disease when the resulting condensate phenotype has a desired modulation of a phenotypic identifier associated with one or more disease-associated factors of the disease.

In other aspects, provided herein is a method of identifying a marker useful for identifying a condensate of interest associated with a disease, the method comprising: (a) assessing a plurality of candidate markers, or precursors thereof, for a level of association with one or more disease-associated factors of the disease; (b) assessing at least one of the plurality of candidate markers for a condensate affinity factor; and (c) identifying the marker from the plurality of candidate markers based on the marker having a desired level of association with the one or more disease-associated factors of the disease and having a desired condensate affinity factor.

The section headings used herein are for organizational purposes only and are not to be construed as limiting the subject matter described. For example, some aspects of the disclosure are presented in a modular fashion, and such presentation is not to be construed as limited the possible combinations of approaches taught herein.

### I. DEFINITIONS

For purposes of interpreting this specification, the following definitions will apply and, whenever appropriate, terms used in the singular will also include the plural and vice versa. In the event that any definition set forth below conflicts with any document incorporated herein by reference, the definition set forth shall control.

As used herein, “condensate” means a non-membrane-encapsulated compartment formed by phase separation of one or more proteins and/or other macromolecules such as nucleic acids (including all stages of phase separation).

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, including but not limited to, methylation, phosphorylation glycosylation, sialylation, or acetylation.

The term “antibody,” and grammatical equivalents thereof, includes full-length antibodies and antigen-binding fragments thereof. A full-length antibody comprises two heavy chains and two light chains. The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multi-specific antibody formed from a portion of an antibody comprising one or more CDRs, a camelized single domain antibody, a nanobody, a domain antibody, a bivalent domain antibody, any other antibody fragment that binds to an antigen but does not comprise a complete full-length antibody structure, or an antibody mimetic (e.g., designed ankyrin repeat proteins (DARPin), affimer, or monobody (ADNECTINS®)). An antigen-binding fragment is capable of binding to the same antigen to which the parent antibody or a parent antibody fragment (e.g., a parent scFv) binds.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### II. METHODS OF IDENTIFYING A CONDENSATE PHENOTYPE AND USES THEREOF

Provided herein are methods of identifying a condensate phenotype associated with a disease. In some embodiments, the identification of a condensate phenotypes enables the identification of a condensate of interest associated with a disease. In some embodiments, the condensate of interest is identified via one or more differences between two or more condensate phenotypes. For example, in some embodiments, the method comprises identifying a condensate of interest as associated with a disease based on a difference between a first condensate phenotype from a first cell model (e.g., a non-disease state cell model or a healthy cell model) and a second condensate phenotype from a second cell model (e.g., a disease cell model), wherein a difference between the first cell model and the second cell model is attributable to one or more disease-associated factors of the disease. In some embodiments, the condensate phenotype associated with a disease comprises one or more phenotypic identifiers of a cell model of the disease.

In some embodiments, the method of identifying a condensate phenotype associated with a disease comprises (a) comparing a first condensate phenotype from a first cell model with a second condensate phenotype from a second cell model, wherein a difference between the first cell model and the second cell model is attributable to one or more disease-associated factors of the disease; and (b) identifying the condensate phenotype associated with a disease based on a difference between the first condensate phenotype and the second condensate phenotype.

In some embodiments, the method of identifying a condensate phenotype associated with a disease comprises (a) comparing a first condensate phenotype from a first cell model with a second condensate phenotype from a second cell model, wherein a difference between the first cell model and the second cell model is attributable to one or more disease-associated factors of the disease, and wherein at least one of the one or more disease-associated factors is introduced to either the first cell model or the second cell model (or both cell models, such as introduced with different disease-associated factors); and (b) identifying the condensate phenotype associated with the disease based on a difference between the first condensate phenotype and the second condensate phenotype. In some embodiments, the one or more disease-associated factors is introduced to the first cell model and the second cell model. In some embodiments, the one or more disease-associated factors introduced to the first cell model and the second cell model are different. For example, a disease-associated factor that contributes less to the disease is introduced to the first cell model, and a disease-associated factor that contributes more to the disease is introduced to the second cell model, and the difference between the first condensate phenotype and the second condensate phenotype is a severity difference, such as a super enlarged condensate vs. an enlarged condensate, while in a non-disease state cell model or a healthy cell model the condensate has a smaller size.

In some embodiments, the method of identifying a condensate phenotype associated with a disease comprises obtaining, such as producing, a first cell model and/or a second cell model. In some embodiments, the method comprises producing a state, such as a treated and/or stimulated state, of a cell model. For example, in some embodiments, the method comprises producing a state of a cell model suitable to obtain a condensate phenotype.

In some embodiments, the method of identifying a condensate phenotype associated with a disease comprises obtaining, such as determining, a first condensate phenotype and/or a second condensate phenotype. In some embodiments, the first condensate phenotype and/or the second condensate phenotype are obtained using an imaging technique.

In some embodiments, the method of identifying a condensate of interest comprises (a) comparing a first condensate phenotype from a first cell model with a second condensate phenotype from a second cell model, wherein a difference between the first cell model and the second cell model is attributable to one or more disease-associated factors of the disease; and (b) identifying the condensate of interest as associated with the disease based on a difference between the first condensate phenotype and the second condensate phenotype for the condensate of interest.

In some embodiments, the method of identifying a condensate of interest comprises (a) comparing a first condensate phenotype from a first cell model with a second condensate phenotype from a second cell model, wherein a difference between the first cell model and the second cell model is attributable to one or more disease-associated factors of the disease, and wherein at least one of the one or more disease-associated factors is introduced to either the first cell model or the second cell model (or both cell models, such as introduced with different disease-associated factors); and (b) identifying the condensate of interest as associated with the disease based on a difference between the first condensate phenotype and the second condensate phenotype for the condensate of interest. In some embodiments, the one or more disease-associated factors is introduced to the first cell model and the second cell model.

In some embodiments, the method of identifying a condensate of interest comprises obtaining, such as producing, a first cell model and/or a second cell model. In some embodiments, the method comprises producing a state, such as a treated and/or stimulated state, of a cell model. For example, in some embodiments, the method comprises producing a state of a cell model suitable to obtain a condensate phenotype and/or a condensate of interest.

In some embodiments, the method of identifying a condensate of interest comprises obtaining, such as determining, a first condensate phenotype and/or a second condensate phenotype. In some embodiments, the first condensate phenotype and/or the second condensate phenotype are obtained using an imaging technique.

Certain features of the methods are described in additional detail in the sections below, including features of, and associated with, diseases and disease-associated factors thereof, cell models, and condensate phenotypes. The modular discussion of such features is not intended to limit the scope of the methods described herein, and using the teachings provided herein one can readily combine various modularly described features to arrive at the full scope of the methods provided herein.

### A. Diseases and Disease-Associated Factors Thereof

The methods of identifying a condensate phenotype and/or a condensate of interest described herein may be applied to evaluate any disease, such as a disease hypothesized to involve a condensate (or lack thereof) that mediates and/or contributes to an aspect of the disease or a disease state. In some embodiments, the presence (or increased level) of an identified condensate of interest mediates or results from a disease. In some embodiments, the absence (or decreased level) of an identified condensate of interest mediates or results from a disease. In some embodiments, the phenotype of an identified condensate of interest mediates or results from a disease. In some embodiments, the identified condensate of interest can serve as a biomarker of a disease, such as for diagnosis and/or to screen for a therapeutic agent. As described herein, in some embodiments, the disease encompasses a disease state, such as a level of progression or severity.

The disease encompassed herein may originate and/or progress due to a single factor or multiple contributing factors. In some embodiments, the disease is caused, at least in part, by a single factor. In some embodiments, the disease is caused, at least in part, by a plurality of factors. In some embodiments, one or more disease-associated factors are associated with the disease. For example, in some embodiments, the one or more disease-associated factors associated with the disease comprise a factor selected from the group consisting of a genetic variation (e.g., a genetic mutation or a genetic variant), post-translational modification variant, exogenous genetic material, level of an endogenous compound, level of an exogenous compound, a physical process (e.g., aging), and a stimulus. In some embodiments, the factor (e.g., genetic variant) has a weak association with the disease, for example in the case of a genetic variant having a log odds ratio of less than or equal to about any of 2, 1.5, 1, or 0.5, or a penetrance higher than 0.95.

In some embodiments, the disease is caused, at least in part, by a genetic factor, such as a genetic variation and/or expression product thereof. In some embodiments, the disease is a Mendelian disease. In some embodiments, the disease is a monogenic disease. In some embodiments, the genetic factor is a genetic variation. In some embodiments, the genetic variation is a genetic variant or genetic mutation, including, but not limited to, a single nucleotide polymorph (SNP) to a larger genetic insertion, deletion, substitution, or repeat expansion, or a combination thereof. In some embodiments, the genetic variation is a point mutation, a termination mutation, a truncation mutation, a mutation that affects splicing, or a frameshift mutation. In some embodiments, the genetic variation is a genetic variant or genetic mutation in a gene, and may be referred to as a “coding variant,” such as a coding SNP. In some embodiments, the genetic variation is a genetic variant or genetic mutation in a non-coding region (including but not limited to promoter, intron, enhancer, intergenic region (IGR), DNase I hypersensitive site (DHS)), and may be referred to as a “non-coding variant,” such as a non-coding SNP. In some embodiments, the genetic variant or genetic mutation is or is within a non-coding RNA (ncRNA). In some embodiments, the genetic variant or genetic mutation in a non-coding region or in an ncRNA affects one or more of i) transcription and/or expression level, ii) post-translational modification, and iii) function of a gene (or gene product) associated with the disease, such as a gene known to cause (directly or indirectly) the disease. In some embodiments, the disease is a polygenic disease. In some embodiments, the genetic factor is a mutation variant. In some embodiments, the genetic factor is a common variant with a minor allele frequency of greater than 1%. In some embodiments, the genetic factor is a rare variant with a minor allele frequency of less than 1%. In some embodiments, the genetic factor is an expression level variant. In some embodiments, the genetic factor is a splicing variant.

In some embodiments, the disease is caused, at least in part, by a post-translational modification variant. In some embodiments, the post-translational modification variant is a polypeptide comprising a post-translational modification. The post-translation modification can be any post-translation modification known in the art (e.g., see “Post translational modifications: an overview,” 2017, PROTEINTECH® blog), including, but not limited to, phosphorylation, methylation, glycosylation, sialylation, acetylation, ADP-ribosylation, famesylation, prenylation, deamidation, proteolysis, geranylgeranylation, hydroxylation, ubiquitylation, nitrosylation, lipidation, O-GlcNAcylation, and UBL-protein conjugation (e.g., sumoylation).

In some embodiments, the disease is caused, at least in part, by an exogenous genetic material. In some embodiments, the exogenous genetic material is from an infectious agent. In some embodiments, the infectious agent is a virus, such as a virus of any one of Orthomyxoviridae, Filoviridae, Flaviviridae, Coronaviridae, adenoviridae, Anelloviridae, Arenaviridae, Astrovididae, Bornaviridae, Bunyaviridae, Caliciviridae, Hepadnaviridae, Hereviridae, Herpesviridae, Papillomaviridae, Paramyxoviridae, Retroviridae, Parvoviridae, Picobirnaviridae, Picobirna, Picornaviridae, Pneumoviridiae, Polyomaviridae, Reoviridae, Rhabdoviridae, Togaviridae, Delta and Poxviridae families. In some embodiments, the infectious agent is a bacterium. In some embodiments, the infectious agent is a fungus. In some embodiments, the infectious agent is a parasite.

In some embodiments, the disease is caused by a viral infection. In some embodiments, the viral infection is an acute infection such as, but not limited to, infection by coronaviruses (e.g., SARS, MERS, SARS-CoV2), enterovirus, hepatitis A and E virus, influenza virus, respiratory virus, or norovirus. In some embodiments, the viral infection is a chronic infection, such as, but not limited to, infection by HIV, hepatitis B, C, and D viruses, or herpesviruses (e.g., cytomegalovirus, herpes simplex virus, varicella zoster virus). In some embodiments, the chronic virus infections may be persistent (such as hepatitis B or C virus infection) and occur over a period of years. In some embodiments, the viral infection is caused by a latent virus. In some embodiments, the latent virus exists in a non-replicating state and the latent virus can be activated, often through stress on the organism, to come out of latency and become an acute, active infection. In some embodiments, the effect of stress on the loss of latency and the induction of an active infection may be regulated through a biological condensate. In some embodiments, for both acute and chronic viral infections, a biological condensate is associated with a virus life cycle. In some embodiments, a biological condensate is associated with an immune response of the host, such as an innate and/or adaptive immune response.

In some embodiments, the disease is caused, at least in part, by an infectious agent, such as a virus, bacterium, fungus, or parasite. In some embodiments, the condensate of interest is involved with the survival of an infectious agent, such as a bacteria, fungi, parasite, or virus, wherein the methods described herein use genes of the infectious agent to assess for condensates of interest according to the methods described herein. For example, in some embodiments, the method comprises predicting the propensity of a gene of the infectious agent to phase separate and/or interact with known condensate proteins. In some embodiments, the introduction of a virus into a cell can be compared to the uninfected cell to identify a condensate phenotype and/or a condensate of interest specific to the infected cells. In some embodiments, cells infected with mycoplasmodium or intracellular parasites can be compared to uninfected cells to identify a condensate phenotype and/or a condensate of interest specific to the infected cells. In some embodiments, the genes of the infectious agent are genes of an infectious agent associated with replication and/or growth.

In some embodiments, the disease is caused, at least in part, by a level (including presence and absence) of an endogenous compound. In some embodiments, the endogenous compound is a hormone. In some embodiments, the endogenous compound is a cytokine. In some embodiments, the endogenous compound is a metabolite of a cellular process, such as a metabolite during nucleic acid biosynthesis, metabolism, apoptosis, endocytosis, citric acid cycle, etc.

In some embodiments, the disease is caused, at least in part, by a level (including presence and absence) of an exogenous compound. In some embodiments, the exogenous compound is selected from the group consisting of a nutrient, a toxic agent, and a toxinogen.

In some embodiments, the disease is caused, at least in part, by a level (including presence and absence) of a physical process, such as stress, aging, physical trauma (such as repeated brain injury), infection and accompanying responses (such as high fever), genetic variations that have deleterious effects on the ability to maintain homeostasis, such as under conditions of cellular stress. In some embodiments, the disease is caused, at least in part, by chronic stress.

In some embodiments, the disease is caused, at least in part, by a level (including presence and absence) of a stimulus. In some embodiments, the stimulus is an environmental stimulus. In some embodiments, the stimulus is selected from the group consisting of temperature, light, sound, pain, pH, and pressure. In some embodiments, the disease is caused, at least in part, by diet. In some embodiments, the stimulus induces stress.

In some embodiments, the disease is a neurodegenerative disease, such as amyotrophic lateral sclerosis (ALS), multiple sclerosis, frontotemporal disorder, Parkinson's disease, and Alzheimer's disease. In some embodiments, the disease is a proliferative disorder, such as cancer. In some embodiments, the disease is an immune disease, such as an autoimmune disease, or an over-active immune response such as cytokine release syndrome (CRS). In some embodiments, the disease is associated with fibrosis formation. In some embodiments, the disease is a cardiac disease, such as familial or non-familial dilated cardiomyopathy (DCM), e.g., DCM directly or indirectly caused by or associated with a mutation in one or more of RNA binding motif protein 20 (RBM20), Desmoplakin (DSP), Desmoglein-2 (DSG2), and alpha-protein kinase 3 (ALPK3). In some embodiments, the disease is associated with a metabolic disorder.

### B. Cell Model Systems

In some aspects, provided herein are cell models useful for the methods of identifying a condensate phenotype and/or a condensate of interest described herein.

In some embodiments, the cell model is a cell model for a disease or a disease state (e.g., a disease cell model), wherein the cell model comprises one or more disease-associated factors attributable to the disease. In some embodiments, provided herein are methods of identifying disease-associated factors useful for designing cell models. In some embodiments, the disease is a multifactorial disease having a plurality of disease-associated factors, wherein a cell model of the disease comprises one or more disease-associated factors attributable to the disease. In some embodiments, the cell model is a cell model for a control or healthy state, wherein the control or healthy cell model does not comprise one or more disease-associated factors attributable to a disease.

In some embodiments, the methods of identifying a condensate phenotype and/or a condensate of interest comprise comparing two cell models. In some embodiments, the methods comprise comparing two cell models, wherein a difference between the two cell models is attributable to one or more disease-associated factors of a disease. In some embodiments, the two cell models are obtained from a single cell model source, e.g., a first cell model is the cell model source and a second cell model is a modified version of the cell model source. In some embodiments, the first cell model is a first modified version of the cell model source and a second cell model is a second modified version of the cell model source. In some embodiments, the two cell models are a non-disease or healthy cell model and a disease cell model. In some embodiments, the two cell models are both disease cell models having a difference attributable to one or more disease-associated factors of the disease, e.g., disease cell models having different disease severities (or disease phenotypes, such as attributed by, e.g., different genetic variants of a gene or different genes). In some embodiments, the one or more disease-associated factors associated with the disease are unknown or not fully known.

The cell model described herein may comprise any number of individual cells, such as in a composition comprising the cell model. In some embodiments, the composition comprises a plurality of cells of the cell model, wherein the plurality of cells are homogeneous.

In some embodiments, the cell model is a transfected cell model (e.g., stably or transiently transfected). In some embodiments, the cell model is a stable cell model. In some embodiments, the cell model is or is derived from an animal cell, such as a mammal cell. In some embodiments, the cell model is or is derived from a human cell. In some embodiments, the cell model is or is derived from a neuron. In some embodiments, the cell model is or is derived from a cancer cell. In some embodiments, the cell model is derived from a cell line, such as HEK293 cells, or a disease cell line, e.g., HeLa cells. In some embodiments, the cell model is or is derived from an induced pluripotent stem cell (iPSC), such as an iPSC-derived motor neuron (iPSC-MN) or an iPSC-derived cardiomyocyte (iPSC-CM), e.g., iCell® Cardiomyocytes. In some embodiments, the cell model is derived from a biopsy or tissue sample, such as from a patient sample, e.g., from a healthy or disease biopsy or tissue sample. In some embodiments, the cell model is derived from a human primary lung fibroblast, such as from a healthy or disease donor lung tissue. In some embodiments, the cell model is derived from a young individual, such as less than 55 years old. In some embodiments, the cell model is derived from an old individual, such as at or older than 55 years old. In some embodiments, the cell model comprises one or more disease-associated factors associated with a disease.

In other aspects, provided herein are methods comprising identifying and/or making a cell model. In some embodiments, the method comprises obtaining a cell model. In some embodiments, the cell model is obtained from a healthy or diseased individual. In some embodiments, one or more disease-associated factors associated with a disease are introduced into a cell model. In some embodiments, the method comprises producing a cell model. In some embodiments, the cell model is treated and/or engineered based on the one or more disease-associated factors associated with a disease.

Techniques for making cell models are well known in the art. In some embodiments, the cell model is produced from a precursor of a cell model via modulating an aspect of the precursor based on one more disease-associated factors associated with a disease. For example, the cell model can be generated by subjecting a precursor cell to stress, such as oxidative stress, or treating a precursor cell with a small molecule compound or hormone. In some embodiments, the cell model is obtained by subjecting a precursor cell to infection, such as an infection by virus, bacteria, fungus, or parasite. In some embodiments, the cell model is produced via knock down or knock out of a genetic feature or expression product thereof, such as by any methods known in the art, e.g., RNAi, TALEN, ZFN, or CRISPR/Cas. In some embodiments, the cell model is provided via knock-in. In some embodiments, the cell model is produced via transfection. In some embodiments, the cell model is transfected with a fusion polypeptide, such as a polypeptide fused to a label, e.g., GFP. In some embodiments, the cell model is transfected with a wild type polypeptide. In some embodiments, the cell model is transfected with a variant polypeptide, such as a mutant polypeptide. In some embodiments, the cell model is transfected to express a level of a gene expression product. In some embodiments, the expression level variant cell model is produced and used in the methods described herein when a gene expression product reaches a pre-determined level.

In some embodiments, the cell model is transfected to express a polypeptide, such as a wild type polypeptide, at a near endogenous level. In some embodiments, the cell model does not express the polypeptide, such as the wild type polypeptide, and the near endogenous level is based on a level of expression of the polypeptide in another cell model. In some embodiments, the cell model is transfected to express a polypeptide with a label, such as a labeled wild type polypeptide, at a near endogenous level, wherein the near endogenous level is based on the level of expression of a respective unlabeled version of the polypeptide. In some embodiments, the cell model has reduced expression of an unlabeled polypeptide, e.g., the cell model comprises a knockout of the unlabeled polypeptide. In some embodiments, the cell model is transfected to express a variant polypeptide, such as a mutant polypeptide (e.g., point mutation, truncation mutation, frameshift mutation, or termination mutation), at a level that is substantially similar to the endogenous expression level of a respective wild type polypeptide of the variant polypeptide. As described herein, in some embodiments, the terms “near endogenous level” or “substantially similar” refer to polypeptide expression levels that are within a 2-fold difference of a measured endogenous level of a polypeptide.

### C. Condensate Phenotypes

In some aspects, provided herein are condensate phenotypes useful for the methods described herein, such as for identifying a condensate of interest associated with a disease. Also provided are methods of obtaining, such as determining, a condensate phenotype.

As described herein, in some embodiments, a condensate phenotype comprises one or more observable or measurable characteristics or phenotypic identifiers associated with a condensate in a cell model. For example, observable or measurable characteristics or phenotypic identifiers associated with a condensate may be determined by imaging a composition comprising cells of a cell model. Observable or measurable characteristics of a condensate phenotype include, but are not limited to, presence (including absence and level/amount), location, distribution, kinetics (such as kinetics of formation or dissolution), morphological (e.g., size, shape, sphericity), material (e.g., fluidity or rigidity), and compositional properties of a condensate.

In some embodiments, the condensate phenotype is characterized by one or more phenotypic identifiers, such as an identifier selected from the group consisting of a condensate presence, absence, level, morphological feature, location, behavior, composition, and material property. In some embodiments, the condensate phenotype comprises the presence of a condensate of interest. In some embodiments, the condensate phenotype comprises the absence (including disappearance or dissolution) of a condensate of interest. In some embodiments, the condensate phenotype comprises the amount of a condensate of interest, including amount based on number of individual condensates and/or a size feature. In some embodiments, the condensate phenotype comprises the amount of a condensate of interest comprising and/or not comprising a component (e.g., marker such as biological marker, or one or more other biomolecules that become components of the condensate under certain conditions). In some embodiments, the condensate phenotype comprises the level (e.g., amount and/or strength) of association of a marker, such as a biomolecule (e.g., polypeptide, DNA, RNA), with a condensate of interest. In some embodiments, the condensate phenotype comprises the level (e.g., amount and/or strength) of association of a first biomolecule (e.g., polypeptide, DNA, RNA) with a second biomolecule in a cell model, wherein one or both of the biomolecules are associated with a condensate of interest, or the two biomolecules associate with different condensates. In some embodiments, the condensate phenotype comprises the abundance (or level of association) of a component of the condensate of interest within the condensate of interest. In some embodiments, the condensate phenotype comprises the location of a condensate of interest or component thereof, such as the subcellular location. For example, a condensate or a component thereof moves to a location where the condensate or component thereof would not normally locate during healthy condition (e.g., translocate to cytoplasm under disease condition). In some embodiments, the condensate phenotype comprises the distribution of a condensate of interest or component thereof (e.g., relative to other cellular organelles, other condensates, or other biomolecules). For example, condensates or components thereof distribute more densely at a subcellular location (e.g., densely distributed around the Golgi apparatus) compared to how they distribute during healthy condition. In some embodiments, the condensate phenotype comprises a morphological feature of a condensate of interest in a cell model, such as size, shape, volume, surface area, and/or sphericity. In some embodiments, the condensate phenotype comprises the number of condensates per cell. In some embodiments, the condensate phenotype comprises the composition of a condensate of interest. In some embodiments, the condensate phenotype comprises the behavior or material property of a condensate of interest, such as dynamic property, liquidity, solidity, or fiber formation. In some embodiments, the condensate phenotype comprises information regarding the kinetics of condensate formation. In some embodiments, the condensate phenotype comprises information regarding the kinetics of condensate dissolution. In some embodiments, the condensate phenotype comprises changes in a phenotypic identifier, such as a formation or dissolution characteristic, in response to an external stimulus.

In some embodiments, the condensate phenotype demonstrates that a condensate of interest is present in, or derived from, a cell model. In some embodiments, the condensate phenotype demonstrates that a condensate of interest is absent in, or not derived from, a cell model.

For example, in some embodiments, obtaining the condensate phenotype comprises measuring an association of a marker with a condensate of interest. In some embodiments, the marker is a biological marker, such as a polypeptide, a DNA, an RNA (coding or non-coding), or any modifications thereof, such as a post-translational modification of a polypeptide (e.g., phosphorylation, glycosylation, O-GlcNAcylation, UBL-protein conjugation (e.g., sumoylation), methylation, sialylation, acetylation, ADP-ribosylation, famesylation, prenylation, deamidation, proteolysis, geranylgeranylation, hydroxylation, ubiquitylation, nitrosylation, lipidation), an epigenetic modification (e.g., histone acetylation or methylation, DNA methylation, etc.), or a modification to a nucleic acid (e.g., RNA capping). In some embodiments, the association of the marker with a condensate of interest is determined using an imaging technique, such as any of the imaging techniques described herein. In some embodiments, the imaging technique comprises labeling the marker, such as expressing the marker as a fluorescence (e.g., GFP)-fusion protein, or via IF-staining.

In some embodiments, the methods described herein comprise obtaining a first condensate phenotype by measuring an association of a first marker (e.g., first biological marker) with the condensate of interest in a first cell model, and obtaining a second condensate phenotype by measuring an association of a second marker (e.g., second biological marker) with the condensate of interest in a second cell model, such as using an imaging technique. In some embodiments, the methods further comprise determining the difference between the first condensate phenotype and the second condensate phenotype. In some embodiments, the first marker and the second marker are the same. In some embodiments, the first marker and the second marker are different.

As discussed herein and the sections below, in some embodiments, the condensate phenotype is determined using a labeling technique. For example, in some embodiments, use of a labeled marker, such as a labeled biological marker, facilitates visualization of a condensate, and determination of an associated observable or measurable characteristic thereof. In some embodiments, the labeled marker associates with the condensate, such as partitions into the condensate. In some embodiments, the labeled marker does not associate with the condensate.

### I. Markers and Biological Markers

In some aspects, provided herein is a marker, such as biological marker, useful for identifying a condensate and/or condensate phenotype. In some embodiments, provided herein is a method of using a marker, such as a biological marker, for assessing a condensate and/or condensate phenotype. In some embodiments, provided herein is a method of identifying a marker, such as a biological marker, useful for assessing or identifying a condensate and/or condensate phenotype.

The markers and biological markers described herein can be any composition (e.g., biomolecule such as polypeptide, DNA (coding or non-coding), RNA (coding or non-coding), hormone, or small molecule compound) known or hypothesized to associate with a condensate in certain (but not necessarily all) states. In some embodiments, the marker (e.g., biological marker) is previously unknown to associate with a condensate, and is identified with any of the methods described herein, such as in silico method. Use of the term marker or biological marker is not intended to imply that the marker or biological marker will always associate with a condensate in, or derived from, a cell model. In some embodiments, the marker, such as the biological marker, associates with a condensate in, or derived from, a cell model. In some embodiments, the marker, such as the biological marker, partitions in a condensate in, or derived from, a cell model. In some embodiments, the marker, such as the biological marker, does not associate with a condensate in, or derived from, a cell model.

In some embodiments, the marker, such as the biological marker, is a macromolecule found or produced in a cell model or composition comprising a cell model. In some embodiments, the marker, such as the biological marker, is a macromolecule that associates with a condensate. In some embodiments, the marker, such as the biological marker, is a macromolecule that dissociates from a condensate. In some embodiments, the marker, such as the biological marker, is a macromolecule that partitions in a condensate. In some embodiments, the marker, such as a biological marker (e.g., non-coding variant), affects one or more of i) transcription and/or expression level (e.g., abundance), ii) post-translational modification, and iii) function (e.g., binding to other molecule (such as protein) or agent (such as compound), incorporation into or dissociation from a condensate) of a macromolecule that associates with, dissociates from, or partitions in a condensate. In some embodiments, the marker, such as a biological marker, is a polypeptide, including a fusion polypeptide (e.g., a GFP fusion polypeptide). In some embodiments, the marker, such as a biological marker, is a nucleic acid, such as a DNA or RNA, either coding or non-coding nucleic acid. In some embodiments, the marker, such as a biological marker, is a gene or a gene product (e.g., RNA or polypeptide). In some embodiments, the marker, such as a biological marker, is an allele (e.g., a single nucleotide polymorphism (SNP) allele, such as a lead SNP), e.g., coding or non-coding SNP. In some embodiments, the marker, such as a biological marker, is a quantitative trait locus (QTL) or a gene associated with QTL, such as an expression QTL (eQTL) or a gene associated with eQTL. In some embodiments, the marker, such as a biological marker, is a lipid. In some embodiments, the marker, such as a biological marker, is a hormone. In some embodiments, the marker is a small molecule compound (e.g., having a molecular weight of 1000 Da or less), such as a dye, or a diagnostic/therapeutic agent.

Generally speaking, a QTL is a genomic locus that correlates with variation of a quantitative trait in a phenotype, e.g., a disease phenotype, and an eQTL is a genomic locus that correlates with variation in expression level of an mRNA. QTLs and eQTLs can be mapped by molecular markers (e.g., SNPs) that correlate with the observed trait or expression variation.

A variation in sequence between two alleles of the same gene within an organism is referred to as an “allelic polymorphism”. The polymorphism can be at a nucleotide within a coding region but, due to the degeneracy of the genetic code, no change in amino acid sequence is encoded. Alternatively, polymorphic sequences can encode a different amino acid at a particular position, but the change in the amino acid does not affect protein function. Polymorphic regions can also be found in non-encoding regions of the gene, or any non-coding region in the genome. In some embodiments, the polymorphism is found in a coding region of the gene or in an untranslated region (e.g., a 5′ UTR, intron, or 3′ UTR) of the gene. “Single nucleotide polymorphism” or “SNP” refers to a polymorphism where each allele differs by the replacement of a single nucleotide in the DNA sequence of the allelic gene. SNPs can also reside in non-coding regions (non-coding SNP). In some cases, the single nucleotide change can alter the structure and/or function of the corresponding gene product (i.e., protein). In some embodiments, a non-coding SNP can affect one or more of i) transcription and/or expression level, ii) post-translational modification, and iii) function of a gene (or gene product; such as incorporation into or dissociation from a condensate). For most SNPs, only two of the four possible nucleotides (A, T, C, or G) are observed. SNPs can be bi-, tri-, or tetra-allelic polymorphisms. However, in humans, tri-allelic and tetra-allelic SNPs are rare, and SNPs are simply referred to as bi-allelic markers.

A set of SNPs can be determined by analyzing publicly available sequence information for genes and identifying alternative forms of a gene having a nucleotide change. Some databases such as Genecards, for example, provide sequences of SNPs. In some embodiments, SNP sites are analyzed for the presence of a restriction enzyme cleavage sequence. In some embodiments, a SNP (coding or non-coding SNP) (e.g., one that associates with a disease, or one or more disease-associated factors of a disease) is identified using a genome-wide association study (GWAS). In some embodiments, a SNP is identified using FUMA, a web-based platform that functionally annotates GWAS findings and prioritizes the most likely causal SNPs and genes (see, e.g., K. Watanabe et al., “Functional mapping and annotation of genetic associations with FUMA,” Nat Commun. 2017; 8(1):1826, the content of which is incorporated herein by reference in its entirety).

Hence, in some embodiments, the marker, such as a candidate marker, is identified by GWAS. In some embodiments, the marker, such as a candidate marker, is identified by FUMA. In some embodiments, the marker is identified by any marker or disease-associated factor (e.g., genetic variant) identification methods described herein (e.g., see section “D. Methods of identifying markers and/or a disease-associated factors” and Example 3 below). In some embodiments, the maker, such as a candidate marker, is identified by QTL mapping, such as eQTL mapping.

The markers and biological markers described herein may be native or non-native (e.g., introduced) to a cell model. For example, in some embodiments, the marker, such as a biological marker, is a polypeptide that is natively expressed in a cell model. In some embodiments, the marker, such as a biological marker, is a polypeptide that is introduced (such as via transfection) to a cell model. In some embodiments, the polypeptide that was introduced to a cell model is natively expressed in, or natively encoded in genetic material of, the cell model. In some embodiments, the polypeptide that was introduced to a cell model is a modified version derived from polypeptide natively expressed in the cell model, such as a polypeptide fused with a label. In some embodiments, the marker, such as a biological marker, is a polypeptide that is natively expressed in a cell model under certain conditions, such as stress, aging, crowding, infection, etc.

In some embodiments, the marker, such as a biological marker, is known to be associated with, such as expressed by, a cell model. In some embodiments, the marker, such as a biological marker, is known to be associated with a disease. In some embodiments, the marker, such as a biological marker, is known to be associated with a disease-associated factor (e.g., causal factor) of a disease. In some embodiments, the marker, such as a biological marker, is a causal factor of a disease.

### 2. Marker Panel

In some embodiments, the methods described herein comprise use of a marker panel comprising a plurality of markers, such as biological markers. In some embodiments, each marker of the marker panel comprises a desired characteristic. In some embodiments, the desired characteristic is based on any one or more of the following: a hypothesized or known association with a condensate, comprises a feature, such as an intrinsically disordered region (IDR), a coiled-coiled domain, or a structured region that is hypothesized or known to associate with a condensate component (such as via protein binding, DNA/RNA binding), is hypothesized or known to be associated with a cellular process, such as a cellular pathway, or is hypothesized or known to be associated with a disease or a disease state, such as having altered expression under the disease state.

In some embodiments, the marker panel identifies a single condensate type, e.g., a condensate type that contains a common macromolecule component. In some embodiments, the marker panel identifies a plurality of condensates (e.g., a pan-condensate marker panel), e.g., certain of the plurality of condensates contains a first macromolecule component and certain of the plurality of condensates does not contain the first macromolecule component. In some embodiments, the marker panel comprises a plurality of markers, such as biological markers, useful for identifying a plurality of condensates, wherein the plurality of condensates comprises two or more types of condensates.

### 3. Imaging Techniques and Image Analysis

In some aspects, the methods described herein comprise use of an imaging technique to visualize a condensate (or lack thereof), such as when assessing a condensate phenotype and/or assessing a condensate of interest. In some embodiments, the methods comprise an image analysis technique useful for assessing a feature of a condensate, such as when determining a condensate phenotype and/or assessing a condensate of interest.

In some embodiments, one or more markers, such as a biological marker, are used to visualize a condensate via an imaging technique. Thus, in some embodiments, the marker, such as a biological marker, comprises a label. In some embodiments, the marker, such as a biological marker, is labeled (such as via an affinity reagent, e.g., an antibody). In some embodiments, the label is selected from the group consisting of a radioactive label, a colorimetric label, a luminescent label, a chemically-reactive label (such as a component moiety used in click chemistry), and a fluorescent label. In some embodiments, the label is a small molecule, such as a compound having a molecular weight of 1000 Da or less. In some embodiments, the label is a small molecule comprising a fluorophore. In some embodiments, the label is associated with, such as covalently or non-covalently, a marker. In some embodiments, the label can be, but not limited to, Halo, dendra2, GFP, RFP, or mCherry.

In some embodiments, the imaging technique comprises use of an immunofluorescence (IF) technique, such as using an affinity label, such as a labeled antibody, that specifically binds to a marker, e.g., a biological marker. In some embodiments, the IF technique comprises subjecting a cell model to an affinity label, such as a labeled antibody, and imaging the cell model. In some embodiments, the method further comprises assessing the captured image for a condensate, such as a condensate of interest, and/or a condensate phenotype.

In some embodiments, the imaging technique comprises use of an in situ hybridization (ISH) technique, e.g., fluorescent ISH (FISH) technique, such as using a nucleic acid probe that specifically binds to a marker, e.g., a biological marker. In some embodiments, the FISH technique comprises subjecting a cell model to a nucleic acid probe, and imaging the cell model. In some embodiments, the method further comprises assessing the captured image for a condensate, such as a condensate of interest, and/or a condensate phenotype.

In some embodiments, the IF and/or FISH technique is performed in a high-throughput manner. For example, in some embodiments, the IF technique comprises assessing a plurality of aliquots of a cell model using one or more affinity labels, such as a labeled antibody. In some embodiments, at least two or more of the aliquots of the cell model are subjected to affinity labels having different specificities, e.g., a first affinity label specific for a first marker and a second affinity label specific for another epitope of the first marker or a second marker. In some embodiments, the aliquots of a cell model are subjected to two or more affinity labels in parallel (such as by subjecting each of two aliquots of a cell model using an affinity label). In some embodiments, the aliquot of a cell model is subjected to two or more affinity labels in series (such as by subjecting the aliquot to a first affinity label, imaging, stripping the first affinity label from the aliquot, and then subjecting the aliquot to a second affinity label). In some embodiments, the aliquot of a cell model is subjected to two or more affinity labels simultaneously. In some embodiments, the aliquots of the cell model are formed in a welled-plate, such as a 384-well plate.

In some embodiments, the FISH technique comprises assessing a plurality of aliquots of a cell model using one or more nucleic acid probes. In some embodiments, at least two or more of the aliquots of the cell model are subjected to nucleic acid probes having different specificities. In some embodiments, the aliquots of a cell model are subjected to two or more nucleic acid probes in parallel (such as by subjecting each of two aliquots of a cell model using a nucleic acid probes). In some embodiments, the aliquot of a cell model is subjected to two or more nucleic acid probes in series (such as by subjecting the aliquot to a first nucleic acid probe, imaging, stripping the nucleic acid probe from the aliquot, and then subjecting the aliquot to a second nucleic acid probe). In some embodiments, the aliquot of a cell model is subjected to two or more nucleic acid probes simultaneously. In some embodiments, the aliquots of the cell model are formed in a welled-plate, such as a 384-well plate.

In some embodiments, the IF and/or FISH technique is performed to identify another marker, such as a biological marker, associated with a condensate or component thereof. For example, in some embodiments, the method comprises subjecting a cell model to at least two affinity labels, wherein a first affinity label is specific for a first marker associated with a condensate, and the second affinity label is specific for another marker. In some embodiments, the method comprises subjecting a cell model to at least two nucleic acid probes, wherein a first nucleic acid probe is specific for a first marker associated with a condensate, and the second nucleic acid probe is specific for another marker. In some embodiments, the method comprises subjecting a cell model to an affinity label and a nucleic acid probe, wherein the affinity label is specific for a first marker associated with a condensate, and the nucleic acid probe is specific for another marker. In some embodiments, the method comprises subjecting a cell model to an affinity label and a nucleic acid probe, wherein the nucleic acid probe is specific for a first marker associated with a condensate, and the affinity label is specific for another marker. In some embodiments, the identification of the second marker associated with the condensate is based on co-localization. In some embodiments, the above methodology is performed in parallel, simultaneously, or in series. In some embodiments, the cell model comprises a first marker comprising a label (e.g., GFP), wherein a second marker is visualized using an IF and/or FISH technique.

In some embodiments, the IF and/or FISH technique is used to assess an association of a marker, such as a biological marker, with a condensate or component thereof. In some embodiments, the IF and/or FISH technique is used to assess an association of a marker, such as a biological marker, with a condensate or component thereof over time, such as via a time-course study. In some embodiments, the IF and/or FISH technique is used to assess an association of a marker, such as a biological marker, with a condensate in the presence of a stimulus, such as a compound, e.g., a therapeutic compound, infection (e.g., viral infection), or an environmental stimulus (e.g., stress).

In some embodiments, the IF technique is a validated IF technique. For example, in some embodiments, the affinity label has been confirmed to associate with an associated marker, including when the marker is partitioned in a condensate. In some embodiments, the affinity label has been confirmed to associate with an associated marker by assessing a cell model having a reduced expression of the marker (such as a knockout or knockdown cell model).

In some embodiments, the methods further comprise use of an additional marker and/or dye to identify a feature of a cell model, such as a boundary of a cell bilayer and/or organelle.

In some embodiments, the methods comprise additional methodology useful for analyzing condensates described herein, such as FRAP or SPT for studying material properties, dynamics, and mobility of condensates. In some embodiments, photo-oligomerizable seeds may be used to map phase-separated fractions (see, e.g., Bracha et al., Cell, 175, 2018).

In some embodiments, the imagining technique comprises use of a microscopy technique (and associated microscopy instrumentation). In some embodiments, the microscopy technique comprises a confocal microscopy technique. In some embodiments, the microscopy technique comprises a fluorescence microscopy technique. In some embodiments, the microscopy technique comprises a high-resolution microscopy technique. In some embodiments, the microscopy technique comprises a stimulated emission depletion (STED) microscopy technique. In some embodiments, the microscopy technique comprises a SoRa super-resolution spinning-disk microscopy technique. In some embodiments, the microscopy technique comprises an electron microscopy technique (such as cryo-EM or cryo-ET). In some embodiments, the microscopy technique comprises a total internal reflection fluorescence (TIRF) microscopy technique.

In some aspects, the methods comprise non-imaging based techniques for determining an aspect of a condensate phenotype. For example, in some embodiments, the method comprises determining the composition of a condensate, such as via a mass spectrometry technique (such as cross-linking mass spectrometry and/or pull-down paired with mass spectrometry), RNA-seq, NMR spectroscopy, or a blot technique (such as a Western blot). For various condensate analysis methods, see, e.g., Basturea, G. N. (“Biological Condensates,” MATER METHODS 2019; 9:2794).

In some embodiments, the methods comprise an analysis technique, such as for assessing an observable or measurable feature of a condensate, for determining a condensate phenotype. In some embodiments, the analysis technique is useful for determining any one or more of a presence (including absence and level, amount, location, and/or distribution), morphological (such as shape, size, volume, surface area, and/or sphericity), material (e.g., kinetics, fluidity, or rigidity), and compositional properties of a condensate. In some embodiments, the analysis technique comprises a manual technique, such as for detecting a condensate and/or condensate phenotype. In some embodiments, the analysis technique comprises an automated or semi-automated analysis technique, such as for detecting a condensate and/or condensate phenotype. In some embodiments, the analysis technique comprises an in silico analysis technique, such as for detecting a condensate and/or condensate phenotype. Certain methods for in silico analysis, such as those incorporating machine learning and deep learning, are known in the art, such as U.S. Pat. No. 10,303,979, which is hereby incorporated by reference herein in its entirety.

D. Methods of Identifying Markers and/or a Disease-Associated Factors

In some aspects, provided herein are methods of identifying a marker (e.g., a biological marker) and/or a disease-associated factor useful for the methods described herein. In some embodiments, the marker is a disease-associated factor. In some embodiments, the marker is useful for the imaging techniques described herein. In some embodiments, the disease-associated factor is useful for obtaining (such as designing and engineering) a cell model described herein.

In embodiments, provided is a method of identifying a marker useful for identifying a condensate of interest associated with a disease, the method comprising: (a) assessing a plurality of candidate markers, or precursors thereof, for a level of association with one or more disease-associated factors of the disease; (b) assessing at least one of the plurality of candidate markers for a condensate affinity factor; and (c) identifying the marker from the plurality of candidate markers based on the marker having a desired level of association with the one or more disease-associated factors of the disease and having a desired condensate affinity factor.

In some embodiments, the method further comprises identifying the one or more disease-associated factors of the disease. In some embodiments, each of the one or more disease-associated factors of the disease is selected from the group consisting of a genetic variant, post-translational modification variant, exogenous genetic material, presence of an endogenous compound, presence of an exogenous compound, a physical process, and environmental stimulus. In some embodiments, the method further comprises identifying a gene or a non-coding variant (e.g., non-coding SNP) associated with the one or more disease-associated factors of the disease. In some embodiments, the method further comprises identifying a gene expression product based on the identified gene, wherein the gene expression product, or a portion thereof, is used to populate the plurality of candidate markers, or precursors thereof. In some embodiments, the level of association of each candidate marker with the one or more disease-associated factors of the disease is based on a disease-causal factor score. In some embodiments, the disease-causal factor score reflects the strength of association of each candidate marker with the one or more disease-associated factors of the disease. In some embodiments, the method further comprises assigning each candidate marker with the disease-causal factor score. In some embodiments, the method comprises ranking a disease-associated factor for a strength of association with a disease (such as to prioritize a list of disease-associated factors to assess via one or more cell models).

In some embodiments, the condensate affinity factor is based on a condensate-association score. In some embodiments, the condensate-association score reflects the strength of association of the candidate marker, or a portion thereof, with any condensate, a specific condensate, and/or a macromolecule associated with a condensate. In some embodiments, the method further comprises assigning the candidate marker with the condensate-association score.

In some embodiments, identifying the marker from the plurality of candidate markers comprises use of a cumulative score that factors in contributions of disease-associated factor(s) and/or condensate affinity factor(s), such as via weighting. In some embodiments, the cumulative score comprises an input that is includes both one or more disease-associated factors and one or more condensate affinity factors. In some embodiments, identifying the marker from the plurality of candidate markers comprises a cumulative score based on the desired level of association with the one or more disease-associated factors of the disease and the desired condensate affinity factor, optionally further with different desired weights in contributing to the cumulative score.

In some embodiments, the marker is a biological marker. In some embodiments, the marker is a gene product, including a variant polypeptide associated therewith. In some embodiments, the marker is a non-coding variant, such as a non-coding RNA (ncRNA) variant, or a genetic variation in a non-coding region (including but not limited to promoter, intron, enhancer, intergenic region (IGR), DNase I hypersensitive site (DHS)). In some embodiments, the marker is a coding SNP or a non-coding SNP.

In some embodiments, the marker is identified in silico.

In some embodiments, the method of identifying a marker (e.g., biological marker) comprises identifying one or more disease-associated factors of a disease. In some embodiments, the method further comprises identifying one or more disease phenotypes (e.g., high blood pressure, neuronal death, or monocyte infiltration). In some embodiments, the disease phenotype is obtained (such as mined or detected) from literature information. In some embodiments, the disease phenotype is obtained (such as mined or detected) via phenotype-phenotype correlations, such as via deep phenotyping cohort studies and/or large biobank datasets (e.g., All of Us, UK Biobank, COPDGene) and related study engines (e.g., Global Biobank Engine, GenoPheno). In some embodiments, the disease phenotype is obtained (such as mined or detected) via genetic overlap of two or more phenotypes, such as obtained via an linkage disequilibrium (LD)-score regression to compute shared heritability or Bayesian colocalization of association statistics of a genomic locus between two or more phenotypes. In some embodiments, each of the one or more disease-associated factors of a disease is selected from the group consisting of a genetic variant, post translational modification variant, exogenous genetic material (e.g., resulting from viral or bacterial infection), presence of an endogenous compound, presence of an exogenous compound, a physical process (e.g., aging), and environmental stimulus. For example, in some embodiments, the method comprises identifying a familial, a rare, or a common genetic variant known or hypothesized to associate with a disease or disease phenotype. Any suitable methods can be used to identify and/or assess one or more disease-associated factors of a disease, such as via genetics-based tools, genome-wide association study (GWAS), linkage testing, rare variant association analysis (or rare variant burden test), predicted loss of function (pLOF) analysis, conditional fine-mapping, expression QTL (eQTL) or splice QTL colocalization, polygenic priority score (PoPS), chromatin interaction mapping, Mendelian analysis (e.g., via OMIM database), and genome annotation enrichment. In some embodiments, the method further comprises assigning a phenotype-causal score for each disease-associated factor, reflecting the strength or level of association or causal relationship of a disease-associated factor for a disease (or disease phenotype). For example, among a plurality of breast cancer disease-associated factors, genetic variations such as BRCA1 and/or BRCA2 may have a higher phenotype-causal score compared to age, pregnancy, and/or lifestyle such as drinking or smoking. For another example, certain genetic variant causes more severe disease phenotype(s), and will have a higher phenotype-causal score compared to other genetic variant(s). Variants statistically linked more to a disease phenotypes of interest have higher phenotype-causal scores. In some embodiments, the method further comprises ranking the plurality of disease-associated factors based on their assigned phenotype-causal scores. In some embodiments, the method further comprises selecting the top, or most desirable, one or more disease-associated factors of a disease. In some embodiments, dimensionality reduction method including, but not limited to a principal component analysis, non-negative matrix factorization, or non-linear dimensionality reduction method, is conducted on a plurality of disease-associated factors to identify the one or more dominating disease-associated factors, e.g., associate more frequently with the disease, or contribute more to disease onset and/or progression. In some embodiments, the dimensionality reduction method is conducted on a plurality of disease phenotypes to identify the one or more dominating disease phenotypes. In some embodiments, the phenotype-causal score of the disease-associated factor is obtained via Mendelian randomization, which uses genetic variation as instrumental variables to investigate the causal relations between disease-associated factors and disease effects (see, e.g., G. Qi and N. Chatterjee, Nat Commun. 2019; 10:1941; N. M. Davies et al. BMJ 2018; 362:k601). In some embodiments, for each of the one or more disease-associated factors, a disease-associated factor vector is obtained, wherein each disease-associated factor vector comprises one or more disease phenotype elements each comprising a metric that measures the severity of a disease phenotype among one or more disease phenotypes of the disease (e.g., blood pressure, neuronal death, monocyte infiltration level), wherein the disease-associated factor vector for each disease-associated factor provides a measurement of the contribution of such disease-associated factor to all disease phenotypes (or disease phenotypes obtained via dimensionality reduction) of the disease, thereby obtaining the phenotype-causal score of the disease-associated factor. In some embodiments, the disease-associated factor vector is further compared to a control factor vector, which comprises one or more control phenotype elements each comprising a metric measuring the corresponding control phenotype (e.g., blood pressure, neuronal survival/amount, monocyte infiltration level) in a non-disease state or healthy organism, wherein the control factor vector provides a measurement of all corresponding control phenotypes (or corresponding control phenotypes obtained via dimensionality reduction) in a non-disease state or healthy organism. In some embodiments, the phenotype-causal score of a disease-associated factor is the difference between the disease-associated factor vector and the control factor vector.

In some embodiments, the method of identifying a disease-associated factor of a disease (e.g., for obtaining a cell model described herein) comprises i) obtaining a plurality of candidate disease-associated factors; ii) obtaining one or more disease phenotypes of the disease; iii) assigning a phenotype-causal score for each of the plurality of candidate disease-associated factors (e.g., using any of the phenotype-causal score obtaining methods described herein), wherein each phenotype-causal score reflects the level of association of each candidate disease-associated factor with the one or more disease phenotypes of the disease; and iv) ranking the plurality of candidate disease-associated factors based on the assigned phenotype-causal score, wherein the top, or most desirable, one or more candidate disease-associated factors are identified as the disease-associated factor of the disease. In some embodiments, the disease-associated factor of the disease is identified in silico.

In some embodiments, the method of identifying a marker (e.g., biological marker) comprises identifying a gene or a non-coding variant (e.g., non-coding SNP) associated with one or more disease-associated factors of a disease. In some embodiments, the method of identifying a marker (e.g., biological marker) comprises identifying a gene or a non-coding variant (e.g., non-coding SNP) associated with one or more disease-associated factors of a disease, from those genes or non-coding variants known or identified to be associated with a condensate or component thereof (e.g., a condensate affinity factor). In some embodiments, a gene or a non-coding variant is identified based on its association with top one or more disease-associated factors of the disease. In some embodiments, a gene or a non-coding variant is identified based on its location (e.g., within any of ±500 kb, ±100 kb, ±50 kb, ±10 kb, ±5 kb, ±1 kb, or ±500 bp) relative to an SNP (e.g., a lead SNP associated with a disease or disease phenotype, such as a lead non-coding SNP or a lead coding SNP). In some embodiments, a gene is identified by identifying some of all SNPs in linkage disequilibrium (LD) with an SNP (e.g., a lead SNP associated with a disease or disease phenotype), and mapping these identified SNPs to corresponding protein-coding genes. In some embodiments, a non-coding variant is identified by identifying some of all SNPs in linkage disequilibrium (LD) with an SNP (e.g., a lead SNP associated with a disease or disease phenotype, such as a lead non-coding SNP or a lead coding SNP). In some embodiments, the method further comprises identifying exonic SNP and/or splicing SNPs from all SNPs in LD with an SNP (e.g., a lead SNP associated with a disease or disease phenotype). In some embodiments, identifying a gene comprises identifying one or more of open reading frame (ORF), regulatory elements (e.g., promoter, enhancer, intronic region), transcription start site, translation start site, RNA spicing site, etc. In some embodiments, identifying a gene comprises identifying a non-coding variant close to the gene (e.g., within any of ±500 kb, ±100 kb, ±50 kb, ±10 kb, ±5 kb, ±1 kb, or ±500 bp of the gene), such as non-coding variants residing in functional non-coding regions such as enhancer elements, DNase hypersensitivity regions, and chromatin marks. In some embodiments, the method further comprises identifying a gene expression product based on the identified gene. In some embodiments, the gene expression product, or a portion thereof, is used as the marker (e.g., biological marker). In some embodiments, the non-coding variant, e.g., the presence of a genetic variation in a non-coding region (such as by PCR or sequencing technology), is used as the marker (e.g., biological marker). In some embodiments, the expression product is an mRNA. In some embodiments, the expression product is a non-coding RNA (ncRNA). In some embodiments, the expression product is a splicing variant. In some embodiments, the expression product is a polypeptide. In some embodiments, the expression product comprises a post-transcriptional or post-translational modification. Any suitable methods can be used to map a disease-associated factor (e.g., genetic variant, mutant protein, viral infection) to a gene, such as using one or more of GWAS tools (e.g., fine-mapping, GitHub, PLINK, BOLT-LMM mixed model association testing, SAIGE (Scalable and Implementation of GEneralized mixed model)), FUMA, molecular integration tools such as colocalization of expression and protein quantitative trait loci (“xQTL”), protein interaction database or prediction algorithms (e.g., IDR prediction tools such as DISOPRED3, ANCHOR, ANCHOR2, IUPred2, alpha-MoRFpred, MoRFpred, fMoRFpred, or MoRFCHiBi; multivalency prediction tools; sequence-function annotation prediction tools), splice site prediction algorithms (e.g., SpliceFinder, NNSplice, MaxEntScan, GeneSplicer, HumanSplicingFinder, or SpliceSiteFinder-like), partitioned heritability, and polygenic priority score (PoPS; see Weeks et al., medRxiv 2020)). In some embodiments, the polygenic priority score is modified to include condensate affinity factors, such as via a gene membership input matrix comprising condensate membership information.

In some embodiments, the method of identifying a marker (e.g., biological marker) comprises assessing one or more genes or non-coding variants (e.g., non-coding SNP) identified to be associated with one or more disease-associated factors of a disease, for a level of association with the one or more disease-associated factors. In some embodiments, the association of a marker (e.g., candidate marker such as biological marker), a gene, or a non-coding variant with one or more disease-associated factors of a disease is based on a disease-causal factor score. In some embodiments, the disease-causal factor score reflects the strength of association of the marker (e.g., biological marker), the gene, or the non-coding variant with one or more disease-associated factors of a disease. In some embodiments, the method of identifying a marker (e.g., biological marker), the gene, or the non-coding variant comprises assigning the marker, the gene, or the non-coding variant with a disease-causal factor score. In some embodiments, the disease-causal factor score is determined based on one or more of i) genomic distance between a disease-associated factor (e.g., causal factor) and a marker (a non-coding variant or a gene), for example, the distance between a viral insertion site (from viral infection) and a gene (e.g., transcription start site of a gene) or a non-coding variant, or the genomic distance between a gene or a non-coding variant and a known genetic variation (i.e., genetic linkage); ii) the frequency of a marker (e.g., genetic variant or gene product) to appear together with a disease-associated factor (e.g., causal factor, such as a familial mutation, smoking) in a disease, for example, the enrichment of the marker in a disease cell type or tissue (e.g., using the method described in H. K. Finucane et al. Nat Genet. 2018; 50(4):621-629); iii) the severity of disease phenotype when a marker (e.g., different genetic variants or different gene products) associates with a disease-associated factor (e.g., causal factor such as viral infection); iv) the strength of interaction of a marker (a non-coding variant or a gene) with a disease-associated factor (e.g., causal factor), such as binding affinity of a marker protein to a disease-associated factor protein (e.g., causal factor protein); v) functional relationship between a marker (a non-coding variant or a gene) and a disease-associated factor (e.g., causal factor) of a disease, such as whether they function in the same signaling pathway, in the same cell type, co-expressed in one or more cell states, and in the same molecular complex, for example, computing the pathway enrichment (such as using DEPICT, see T. H. Pers et al. Nat Commun. 2015; 6:589) and/or a protein network enrichment (such as using GWAS Summary Statistics and/or Disease Association Protein-Protein Link Evaluator (DAPPLE; see E. J. Roissin et al. PLoS Genet. 2011; 7(1):e1001273)) of a marker, a non-coding variant, or a gene and optionally prioritizing, or whether the non-coding variant affects one or more of a) transcription and/or expression level, b) post-translational modification, and c) function of the disease-associated factor (e.g., a gene or gene product), such as a gene known to cause (directly or indirectly) the disease; etc. In some embodiments, the method further comprises ranking a plurality of markers (such as candidate markers, e.g., biological markers), genes, or non-coding variants based on their assigned disease-causal factor scores. In some embodiments, the method further comprises selecting one or more top-ranked markers (e.g., biological markers), genes, or non-coding variants based on their assigned disease-causal factor scores.

In some embodiments, the method of identifying a marker (e.g., biological marker) comprises identifying a gene or a non-coding variant (e.g., non-coding SNP) associated with a condensate or component thereof (e.g., a condensate affinity factor). In some embodiments, the method of identifying a marker (e.g., biological marker) comprises assessing one or more genes or non-coding variants for a level of association with a condensate or component thereof (e.g., a condensate affinity factor). In some embodiments, the method of identifying a marker (e.g., biological marker) comprises assessing one or more genes or non-coding variants (e.g., non-coding SNP) known or identified to be associated with one or more disease-associated factors of a disease, for a level of association with a condensate or component thereof (e.g., a condensate affinity factor). In some embodiments, the association of a marker (e.g., biological marker), a gene, or a non-coding variant with a condensate or component thereof (e.g., a condensate affinity factor) is based on a condensate-association score. In some embodiments, the condensate-association score reflects the strength of association of the marker (e.g., non-coding variant or coding variant), or a portion thereof, with any condensate, a specific condensate, and/or a macromolecule (e.g., polypeptide, RNA, DNA) associated with a condensate. In some embodiments, the condensate-association score is a composite score of a condensate affinity score and a condensate function score. In some embodiments, the condensate affinity score and the condensate function score are each given a weight (e.g., 40% vs. 60%) to obtain the composite condensate-association score. In some embodiments, the method of identifying a marker (e.g., biological marker) comprises assigning the marker with a condensate-association score. In some embodiments, the condensate-association score is determined based on factors comprising one or more of (optionally each with a desired weight in contributing to the condensate-association score) (i) partition characteristics (e.g., partition coefficient) of a marker or a gene product; (ii) binding affinity of a marker (or a gene or gene product) to a condensate component or a macromolecule (e.g., polypeptide, RNA, DNA) associated with a condensate (e.g., the macromolecule becomes a condensate component under disease or stress state); (iii) protein structure or domain of a marker or a gene product, such as one or more of presence/absence/amount/degree of disorder region such as IDR (e.g., using Predictor of Natural Disordered Regions (PONDR®)), presence/absence/amount of condensate-favoring motifs such as coiled-coil domain and its strength in mediating protein aggregation, and presence/absence/amount of interacting domains to achieve interaction multivalency; iv) the level of association of a marker (e.g., a gene or gene product, or non-coding variant) with a condensate function; v) condensate or protein image analysis of a marker (or a gene or gene product), such as based on data from Human Protein Atlas (HPA) to determine if a protein forms a condensate-like structure; vi) predicted condensate formation ability (e.g., score); etc. In some embodiments, the level of association of a marker (e.g., a gene or gene product, or non-coding variant) with a condensate function is determined based on a condensate function score, which is determined based on one or more factors of (optionally each with a desired weight in contributing to the condensate function score) whether a genetic variation corresponding to the marker (or a portion thereof), the gene, or the non-coding variant associated with one or more disease-associated factors (i) is within a disordered region (e.g., an IDR) and/or subject to post-translational modification (e.g., phosphorylation, ubiquitination, sumoylation, methylation, or acetylation); (ii) affects splicing of the marker or the gene (e.g., using tools such as SpliceAI, SpliceFinder, etc.); (iii) affects the chromatin state (e.g., histone modification, nucleosome density and/or distribution, etc., such as using DeepSEA which detect epigenetic modifications) near the gene or the non-coding variant identified to be associated with one or more disease-associated factors (e.g., within any of ±500 kb, ±100 kb, ±50 kb, ±10 kb, ±5 kb, ±1 kb, or ±500 bp of the gene or the non-coding variant); iv) affects expression of the gene identified to be associated with one or more disease-associated factors, which can, for example, indirectly impact a condensate via the abundance of the gene product; etc. In some embodiments, the condensate affinity score is determined based on one or more factors of (optionally each with a desired weight in contributing to the condensate affinity score) (i) the presence/absence/amount/degree of disorder region such as IDR (e.g., using PONDR®), (ii) the presence/absence/amount/degree of condensate-favoring motifs such as coiled-coil domain, and (iii) the presence/absence/amount/valency of interacting domains to achieve interaction multivalency in the marker or the gene product. In some embodiments, the method further comprises ranking a plurality of markers (such as candidate markers, e.g., biological markers) based on their assigned condensate-association scores. In some embodiments, the method further comprises selecting one or more top-ranked markers (e.g., biological markers) based on their assigned condensate-association scores.

In some embodiments, the method of identifying a marker (e.g., biological marker) comprises assessing one or more genes identified to be associated with one or more disease-associated factors of a disease by a causal strength of the dosage relationship between the gene (or gene product) and the disease. In some embodiments, the causal strength of the dosage is based on an effect-size of dosage score, which is calculated as a ratio between gene (or gene product) abundance to disease effect size using Mendelian randomization (MR; see, e.g., G. Qi and N. Chatterjee, Nat Commun. 2019; 10:1941; N. M. Davies et al. BMJ 2018; 362:k601). In some embodiments, the method of identifying a marker (e.g., biological marker) comprises assigning the marker with an effect-size of dosage score. In some embodiments, the method further comprises ranking a plurality of markers (such as candidate markers, e.g., biological markers) based on their assigned effect-size of dosage scores. In some embodiments, the method further comprises selecting one or more top-ranked markers (e.g., biological markers) based on their assigned effect-size of dosage scores.

In some embodiments, the method of identifying a marker (e.g., biological marker) comprises identifying a maker based on the marker having a desired level of association with one or more disease-associated factors of the disease, a desired condensate affinity factor, and optionally a desired causal strength of the dosage. In some embodiments, such combination of desired levels is based on a cumulative score comprising weighted disease-causal factor score, condensate-association score, and optionally effect-size of dosage score. Such cumulative score informs on each disease-associated factor or marker: the degree to which the marker is a condensate gene and/or the disease variant acts through a condensate mechanism, the degree to which human molecular data corroborates the hypothesis, and optionally magnitude of the effect.

In some embodiments, the method of identifying a marker (e.g., biological marker) comprises selecting the marker from a plurality of candidate markers based on the strength of association of the marker with: (i) one or more disease-associated factors of a disease; and (ii) a condensate or component thereof. In some embodiments, selecting a marker (e.g., biological marker) from a plurality of candidate markers comprises any one of the following steps: a-i) assigning a disease-causal factor score for each of the plurality of candidate markers, wherein each of the plurality of candidate markers has an association with a condensate or component thereof; and a-ii) ranking the plurality of candidate markers based on their assigned disease-causal factor scores; wherein the top ranked (one or more) candidate marker(s) is selected as the final marker(s); or b-i) assigning a condensate-association score for each of the plurality of candidate markers, wherein each of the plurality of candidate markers has an association with one or more disease-associated factors of a disease; and b-ii) ranking the plurality of candidate markers based on their assigned condensate-association scores; wherein the top ranked (one or more) candidate marker(s) is selected as the final marker(s); or c-i) assigning a disease-causal factor score for each of the plurality of candidate markers; c-ii) assigning a condensate-association score for each of the plurality of candidate markers; c-iii) assigning a cumulative score for each of the plurality of candidate markers based on the assigned disease-causal factor score and the assigned condensate-association score; and c-iv) ranking the plurality of candidate markers based on their assigned cumulative scores; wherein the top ranked (one or more) candidate marker(s) is selected as the final marker(s). In some embodiments, the method further comprises assigning, ranking, and selecting candidate marker(s) or final maker(s) for the effect-size of dosage score(s), or the cumulative score(s) taking into account of the effect-size of dosage score(s).

In some embodiments, provided herein is a method of identifying a marker, such as a gene or gene product, e.g., a wild type or variant polypeptide, or a non-coding variant (e.g., non-coding SNP) useful for identifying a condensate of interest associated with a disease, the method comprising: (a) assessing a plurality of candidate markers, or precursors thereof, for a level of association with the disease; (b) assessing at least one of the plurality of candidate markers for a condensate affinity factor; and (c) identifying the marker from the plurality of candidate markers based on the marker having a level of association with the disease and having one or more condensate affinity factors, wherein the identifying is performed using a cumulative score that factors in contributions of level of association with the disease (e.g., including the level of association with one or more disease-associated factors of the disease) and/or one or more of the condensate affinity factor(s).

In some embodiments, provided herein is a method of identifying a marker, such as a gene or gene product, e.g., a wild type or variant polypeptide, or a non-coding variant, useful for identifying a condensate of interest associated with a disease, the method comprising: (a) assessing a plurality of candidate markers, or precursors thereof, for a level of association with one or more disease-associated factors of the disease; (b) assessing at least one of the plurality of candidate markers for a condensate affinity factor; and (c) identifying the marker from the plurality of candidate markers based on the marker having a level of association with the one or more disease-associated factors of the disease and having one or more condensate affinity factors, wherein the identifying is performed using a cumulative score that factors in contributions of one or more of the disease-associated factor(s) and/or one or more of the condensate affinity factor(s).

In some embodiments, the cumulative score weights the factors assessed therein. In some embodiments, the cumulative score provides a list of candidate markers that are further assessed via one or more condensate affinity factors. In some embodiments, the plurality of candidate markers is identified based on a GWAS analysis pertaining to a condition such as a disease. In some embodiments, the one or more disease-associated factors are based on (a) condensate-focused polygenic gene features (PoPS or modified PoPS), (b) PoPS, (c) Mendelian gene factors, (d) rare variant burden, (e) mapping protein-coding genes by SNPs in linkage disequilibrium with independent significant SNP, (f) eQTL colocalization across tissue(s), and (g) chromatin interaction (e.g., via Hi-C or 3C mapping). In some embodiments, the one or more condensate affinity factors are based on a probability of phase-separation formation (e.g., DeepPhase score), predicted condensate formation (e.g., Pscore), presence of an intrinsically disordered region (IDR region) or a fraction of an IDR region, known association with a condensate, and protein image from the Human Protein Atlas (EPA).

In some embodiments, provided herein is a method of identifying a marker, such as a gene or gene product, e.g., a wild type or variant polypeptide, or a non-coding variant, useful for identifying a condensate of interest associated with a disease, the method comprising: (a) obtaining a plurality of candidate markers associated with a disease, such as via a GWAS analysis; (b) assessing the plurality of candidate markers, or precursors thereof, for a level of association with one or more disease-associated factors of the disease, wherein, in some embodiments, a disease-associated factor may incorporate one or more condensate affinity factors (such as via a modified PoPS score including one or more condensate affinity factors); (c) assessing at least one of the plurality of candidate markers for a condensate affinity factor; and (d) identifying the marker from the plurality of candidate markers based on the marker having a level of association with the one or more disease-associated factors of the disease and having one or more condensate affinity factors, wherein the identifying is performed using a cumulative score that factors in contributions of one or more of the disease-associated factor(s) and/or one or more of the condensate affinity factor(s).

In some embodiments, the method comprises use of a scoring technique to prioritize the plurality of candidate markers, or precursors thereof, based on the one or more disease-associated factors of the disease, wherein, in some embodiments, a disease-associated factor may incorporate one or more condensate affinity factors (such as via a modified PoPS score including one or more condensate affinity factors), e.g., a gene prioritization score. In some embodiments, the cumulative score weights the factors assessed therein. In some embodiments, the cumulative score provides a list of candidate markers that are further assessed via one or more condensate affinity factors. In some embodiments, the plurality of candidate markers is identified based on a GWAS analysis pertaining to a condition such as a disease. In some embodiments, the one or more disease-associated factors are based on (a) condensate-focused PoPS, (b) PoPS, (c) Mendelian gene factors, (d) rare variant burden, (e) mapping protein-coding genes by SNPs in linkage disequilibrium with independent significant SNP, (f) eQTL colocalization across tissue(s), and (g) chromatin interaction (e.g., via Hi-C or 3C mapping). In some embodiments, the one or more condensate affinity factors are based on a probability of phase-separation formation (e.g., DeepPhase score), predicted condensate formation (e.g., Pscore), presence of an intrinsically disordered region (IDR region) or a fraction of an IDR region, known association with a condensate, and protein image from the Human Protein Atlas (EPA).

In some embodiments, the marker (e.g., biological marker) is identified in silico, such as using one or more knowledge, databases, algorithms, methods in bioinformatics, transcriptomics, proteomics, genetics, genomics, epigenomics, system biology, system medicine or pharmacology, machine learning, deep learning, etc. In some embodiments, the method further comprises verifying the association of the identified/selected marker (e.g., biological marker) with: (i) the one or more disease-associated factors of the disease; (ii) the condensate or the component thereof; and optionally (iii) disease effect size. Any suitable methods known in the art and/or described herein can be used for such verification. For example, mutating an identified biological marker (e.g., gene or non-coding variant) in a cell or an organism and examining cell function and/or disease phenotype. For example, inhibiting the function of an identified biological marker (e.g., kinase) in a disease cell model or disease organism and examining the restoration of cell function and/or alleviation/elimination of the disease. For another example, conducting IF staining on a condensate in a disease cell model and examining the presence of such biological marker within the condensate. For another example, forming in vitro condensates in the presence of the identified marker and examining the association of such marker with the in vitro condensates.

### E. Comparing Condensate Phenotypes

In some aspects, the method comprises comparing condensate phenotypes to identify a difference between the condensate phenotypes.

In some embodiments, the method comprises use of any quantitative and/or qualitative image analysis methods to determine the difference between condensate phenotypes. In some embodiments, the method comprises use of a manual image analysis method to determine the difference between condensate phenotypes. In some embodiments, the method comprises use of a semi-automated or automated image analysis method to determine the difference between condensate phenotypes. In some embodiments, the semi-automated or automated image analysis method further comprises manual validation to determine the difference between condensate phenotypes.

In some embodiments, the method for comparing condensate phenotypes to identify a difference between the condensate phenotypes comprises use of a deep convolutional neural network, such as a trained deep convolutional neural network. In some embodiments, the method comprises use of a supervised, weakly supervised, or unsupervised algorithm. In some embodiments, the algorithm utilizes weakly supervised learning of single-cell features embeddings. In some embodiments, the method comprises use of multiple instance learning, such as to combine a convolutional neural network and multiple instance learning. In some embodiments, the method comprises conventional single cell feature extraction (e.g., pixel intensity, shape, texture, and/or colocalization characteristics) from segmentation of one or more biological entities (e.g., nucleus, full cell, and/or condensate), such as for use in a qualitative and quantitative analysis. See, e.g., Caicedo et al., bioRxiv, 2018; Cuccarese et al., bioRxiv, 2020; Kraus et al., Bioinformatics, 32, 2016; and McQuin et al., PLoS Biol, 16, 2018, which are incorporated herein by reference in their entirety.

In some embodiments, the methods comprise determining a difference between condensate phenotypes obtained from a first cell model and a second cell model, wherein a difference between the first cell model and the second cell model is attributable to one or more disease-associated factors of a disease. In some embodiments, the causal factor of the disease is unknown or not fully known.

In some embodiments, determining the difference between a first condensate phenotype and a second condensate phenotype comprises a qualitative assessment, such as condensate fluidity, shape or sphericity of a condensate, the function of a condensate or component thereof, or biological activities (e.g., cell signaling, cell function) involving a condensate or component thereof. In some embodiments, determining a difference between a first condensate phenotype and a second condensate phenotype comprises a quantitative assessment, such as fluorescence intensity, condensate fluidity, shape and/or size of a condensate, number of condensates, distribution density of condensates within an area, etc. In some embodiments, determining the difference(s) between a first condensate phenotype and a second condensate phenotype uses one or more of the phenotype identification methods described herein, such as imaging technique (including image processing technique), IF, FRAP, MS, etc. In some embodiments, determining the difference(s) between a first condensate phenotype and a second condensate phenotype comprises an in silico technique, such as including statistics, bioinformatics, a machine learning and/or deep learning technique, or any available algorithms suitable for protein/protein complex/aggregate/membraneless granule studies.

In some embodiments, comparing condensate phenotypes between two cell models comprises comparing the level of association of a marker (e.g., biological marker) with a condensate of interest in, or derived from, a disease cell model (e.g., diseased cardiomyocyte model) as compared to (i) the level of association of the marker (e.g., biological marker) with the condensate of interest in, or derived from, a reference cell model (e.g., healthy cell, or an irrelevant disease cell model such as cancel cell model); or (ii) the level of association of a counterpart of the marker (e.g., genetic variant) with the condensate of interest in, or derived from, the disease cell model. In some embodiments, detection of the difference in the level of association identifies the condensate of interest as associated with the disease.

In some embodiments, the method of identifying a condensate of interest associated with a disease comprises detecting a modulation in a level of association of a marker (e.g., biological marker) with the condensate of interest in, or derived from, a disease cell model as compared to: (i) the level of association of the marker (e.g., biological marker) with the condensate of interest in, or derived from, a reference cell model (e.g., healthy cell, or an irrelevant disease cell model); or (ii) the level of association of a counterpart of the marker (e.g., biological marker, such as genetic variant) with the condensate of interest in, or derived from, the disease cell model, wherein detection of the modulation identifies the condensate of interest as associated with the disease. In some embodiments, the marker (e.g., biological marker) is selected based on having an association with: (i) one or more disease-associated factors of the disease; (ii) a condensate or a component thereof; and optionally (iii) disease effect size. In some embodiments, the method further comprises identifying the marker (e.g., biological marker) based on having an association with: (i) the one or more disease-associated factors of the disease; (ii) the condensate or the component thereof; and optionally (iii) disease effect size.

### F. Condensates

The methods provided herein are useful for evaluating a diverse array of condensates and condensate types.

In some embodiments, the condensate, such as the condensate of interest, is present in, or derived from, a cell model. In some embodiments, the condensate, such as the condensate of interest, is a cellular condensate. In some embodiments, the condensate, such as the condensate of interest, is localized in a specific location of a cell, such as an organelle, e.g., the nucleus. In some embodiments, the condensate, such as the condensate of interest, is not localized in a specific location of a cell, such as diffusing around the entire cell, or throughout the cytosol. In some embodiments, the condensate, such as the condensate of interest, is an extracellular condensate.

In some embodiments, the condensate, such as the condensate of interest, is only found in a disease cell model. In some embodiments, the condensate, such as the condensate of interest, is only found a healthy cell model. In some embodiments, the condensate of interest is present in, or derived from, the first cell model. In some embodiments, the condensate of interest is absent in, or not derived from, the second cell model. In some embodiments, the condensate of interest is absent in, or not derived from, the first cell model. In some embodiments, the condensate of interest is present in, or derived from, the second cell model.

The condensate, such as the condensate of interest, can be any condensate known in the art. In some embodiments, the condensate, such as the condensate of interest, belongs to a condensate type selected from the group consisting of a stress granule, cleavage body, p-granule, histone locus body, multivesicular body, neuronal RNA granule, nuclear gem, nuclear pore, nuclear speckle, nuclear stress body, nucleolus, Oct1/PTF/transcription (OPT) domain, paraspeckle, perinucleolar compartment, PML nuclear body, PML oncogenic domain, polycomb body, processing body, Sam68 nuclear body, and splicing speckle. In some embodiments, the condensate, such as the condensate of interest, is previously unknown and identified by any methods described herein.

In some embodiments, the condensate of interest is associated with familial DCM, such as present in familial DCM patients, or has a different condensate phenotype in familial DCM patients compared to healthy individuals. In some embodiments, the condensate of interest is an RBM20 condensate, a DSP condensate, a DSG2 condensate, or an ALPK3 condensate.

### G. Additional Methods

In some embodiments, the method of identifying a condensate of interest comprises detecting a modulation in a level of association of a marker (e.g., biological marker) with the condensate of interest, such as a marker identified/selected using any of the methods described herein. In some embodiments, detecting the modulation in the level of association of the biological marker with the condensate of interest is performed via an imaging technique, such as any of the imaging techniques described herein.

In some embodiments, the method of identifying a condensate of interest further comprises determining: the level of association of a marker (e.g., biological marker), such as a marker identified/selected using any of the methods described herein, with the condensate of interest in, or derived from, a reference cell model; or the level of association of a counterpart of the marker (e.g., biological marker), such as a marker identified/selected using any of the methods described herein, with the condensate of interest in, or derived from, a disease cell model. In some embodiments, the counterpart of the biological marker is a genetic variant, splicing variant, or post-translational modification variant of the biological marker.

In some embodiments, the method of identifying a condensate of interest further comprises verifying the condensate of interest as associated with a disease. Any suitable methods known in the art and/or described herein can be used for such verification. For example, inducing the formation of a disease condensate in a healthy cell model or organism (e.g., knock-out endogenous marker protein expression and inducing the expression of a counterpart mutant marker protein) and examining cell function and/or disease phenotype. For another example, using a small molecule compound to cause dissociation of a condensate of interest in a disease cell model or disease organism, and examining the restoration of cell function and/or alleviation/elimination of the disease.

In other aspects, provided herein is a method of identifying a condensate of interest associated with a disease, the method comprising: (a) comparing a first condensate phenotype from a first cell model with a second condensate phenotype from a second cell model, wherein the first condensate phenotype and the second condensate phenotype are obtained using a marker identified using any one of the methods described herein, wherein a difference between the first cell model and the second cell model is attributable to one or more disease-associated factors of the disease, and wherein at least one of the one or more disease-associated factors is introduced to either the first cell model or the second cell model; and (b) identifying the condensate of interest as associated with the disease based on a difference between the first condensate phenotype and the second condensate phenotype for the condensate of interest.

### H. Exemplary Methods of Identifying a Condensate of Interest

In some aspects, provided is a method of identifying a condensate phenotype associated with a disease, the method comprising (a) comparing a first condensate phenotype from a first cell model with a second condensate phenotype from a second cell model, wherein a difference between the first cell model and the second cell model is attributable to one or more disease-associated factors; and (b) identifying the second condensate phenotype as a condensate phenotype associated with a disease based on a difference between the first condensate phenotype and the second condensate phenotype. In some embodiments, a condensate of interest associated with the disease is identified from the condensate phenotype associated with the disease. In some embodiments, provided is a method of identifying a condensate of interest associated with a disease, the method comprising: (a) comparing a first condensate phenotype from a first cell model with a second condensate phenotype from a second cell model, wherein a difference between the first cell model and the second cell model is attributable to one or more disease-associated factors; and (b) identifying the condensate of interest as associated with the disease based on a difference between the first condensate phenotype and the second condensate phenotype for the condensate of interest. In some embodiments, the condensate phenotype, such as the first condensate phenotype or the second condensate phenotype, is obtained using a marker panel comprising markers known or thought to associate with a condensate. In some embodiments, the condensate phenotype is obtained, such as determined, using an imaging technique designed for visualizing the markers of the marker panel. In some embodiments, the imaging technique comprises labeling a marker using any one or more of IF, ISH (such as FISH), gene fusion (e.g., GFP labeling), or a dye that is specific for a marker and/or a condensate. In some embodiments, the imaging technique comprises an automated image analysis. In some embodiments, the disease-associated factor of the cell model is identified and/or selecting using an in silico method described herein.

In some aspects, provided is a method of identifying a condensate phenotype associated with a disease, the method comprising (a) comparing a first condensate phenotype from a first cell model with a second condensate phenotype from a second cell model, wherein a difference between the first cell model and the second cell model is attributable to one or more disease-associated factors; and (b) identifying the second condensate phenotype as a condensate phenotype associated with a disease based on a difference between the first condensate phenotype and the second condensate phenotype. In some embodiments, a condensate of interest associated with the disease is identified from the condensate phenotype associated with the disease. In some embodiments, provided is a method of identifying a condensate of interest associated with a disease, the method comprising: (a) comparing a first condensate phenotype from a first cell model with a second condensate phenotype from a second cell model, wherein a difference between the first cell model and the second cell model is attributable to one or more disease-associated factors; and (b) identifying the condensate of interest as associated with the disease based on a difference between the first condensate phenotype and the second condensate phenotype for the condensate of interest. In some embodiments, the condensate phenotype, such as the first condensate phenotype or the second condensate phenotype, is obtained using a marker identified using an in silico method described herein. In some embodiments, the condensate phenotype is obtained, such as determined, using an imaging technique designed for visualizing the markers of the marker panel. In some embodiments, the imaging technique comprises labeling a marker using any one or more of IF, ISH (such as FISH), gene fusion (e.g., GFP labeling), or a dye that is specific for a marker and/or a condensate. In some embodiments, the imaging technique comprises an automated image analysis. In some embodiments, the disease-associated factor of the cell model is identified and/or selecting using an in silico method described herein.

In some aspects, provided is a method of identifying a condensate phenotype associated with a disease, the method comprising (a) comparing a set of condensate phenotypes, wherein each condensate phenotype of the set of condensate phenotypes is from a respective cell model, and wherein differences between the respective cell models is attributable to one or more disease-associated factors; and (b) identifying at least two of the condensate phenotypes of the set of condensate phenotypes as associated with a disease based on a convergent difference between the at least two of the condensate phenotypes and the other condensate phenotypes of the set of condensate phenotypes. In some embodiments, each of the respective cell models comprises a unique combination of the one or more disease-associated factors. In some embodiments, a condensate of interest associated with the disease is identified based on the convergent difference between the at least two of the condensate phenotypes and the other condensate phenotypes of the set of condensate phenotypes. In some embodiments, the condensate phenotype is obtained using a marker panel comprising markers known or thought to associate with a condensate. In some embodiments, the condensate phenotype, such as the first condensate phenotype or the second condensate phenotype, is obtained using a marker identified using an in silico method described herein. In some embodiments, the condensate phenotype is obtained, such as determined, using an imaging technique designed for visualizing the markers of the marker panel. In some embodiments, the imaging technique comprises labeling a marker using any one or more of IF, ISH (such as FISH), gene fusion (e.g., GFP labeling), or a dye that is specific for a marker and/or a condensate. In some embodiments, the imaging technique comprises an automated image analysis. In some embodiments, the disease-associated factor of the cell model is identified and/or selecting using an in silico method described herein.

In some embodiments, provided is a method of identifying a condensate of interest associated with a disease, the method comprising: (a) comparing a set of condensate phenotypes, wherein each condensate phenotype of the set of condensate phenotypes is from a respective cell model, and wherein differences between the respective cell models is attributable to one or more disease-associated factors; and (b) identifying the condensate of interest as associated with the disease based on a convergent difference identified in at least two of the condensate phenotypes of the set of condensate phenotypes. In some embodiments, the condensate phenotype is obtained using a marker panel comprising markers known or thought to associate with a condensate. In some embodiments, the condensate phenotype, such as the first condensate phenotype or the second condensate phenotype, is obtained using a marker identified using an in silico method described herein. In some embodiments, the condensate phenotype is obtained, such as determined, using an imaging technique designed for visualizing the markers of the marker panel. In some embodiments, the imaging technique comprises labeling a marker using any one or more of IF, ISH (such as FISH), gene fusion (e.g., GFP labeling), or a dye that is specific for a marker and/or a condensate. In some embodiments, the imaging technique comprises an automated image analysis. In some embodiments, the disease-associated factor of the cell model is identified and/or selecting using an in silico method described herein.

### III. FURTHER ASPECTS ENABLED BY THE METHODS DISCLOSED HEREIN

In other aspects, provided herein are further methods and aspects enabled by the identification of a condensate phenotype, condensate of interest, cell model, and a marker, and associated methods described herein.

In some embodiments, provided herein is a method of identifying a compound that modulates a condensate phenotype, the method comprising: (a) admixing the compound and a composition comprising a cell model; and (b) obtaining a resulting condensate phenotype of the composition, wherein a difference between the resulting condensate phenotype and a reference condensate phenotype identifies the compound as modulating the condensate phenotype. In some embodiments, the cell model comprises a disease-associated factor. In some embodiments, the disease-associated factor is identified using a method described herein. In some embodiments, the reference condensate phenotype is a condensate phenotype of a reference cell model, wherein a difference between the cell model and the reference cell model is attributable to one or more disease-associated factors. In some embodiments, the resulting condensate phenotype and/or the reference condensate phenotype are imaged using a marker, such as a biological marker. In some embodiments, the resulting condensate phenotype and/or the reference condensate phenotype are imaged using a marker panel. In some embodiments, the marker is identified using a method described herein.

In some embodiments, provided herein is a method of identifying a compound useful for treating a disease, the method comprising: (a) admixing the compound and a composition comprising a cell model; (b) obtaining a resulting condensate phenotype of the composition, wherein the compound is identified as useful for treating the disease when the resulting condensate phenotype has a desired modulation of a phenotypic identifier associated with one or more disease-associated factors of the disease. In some embodiments, the cell model comprises a disease-associated factor. In some embodiments, the disease-associated factor is identified using a method described herein. In some embodiments, the reference condensate phenotype is a condensate phenotype of a reference cell model, wherein a difference between the cell model and the reference cell model is attributable to one or more disease-associated factors. In some embodiments, the resulting condensate phenotype and/or the reference condensate phenotype are imaged using a marker, such as a biological marker. In some embodiments, the resulting condensate phenotype and/or the reference condensate phenotype are imaged using a marker panel. In some embodiments, the marker is identified using a method described herein.

In other aspects, provided herein is a method of identifying a biological component associated with a disease, the method comprising identifying a condensate of interest according to any one of the methods described herein, and identifying the biological component based on an association with the condensate of interest or a component thereof. In some embodiments, the biological component is identified based on partitioning into the condensate of interest. In some embodiments, the biological component is identified based on modulating the partitioning of another component into the condensate of interest.

In other aspects, provided herein is a method of identifying one or more interactions of a test compound, or a portion thereof, and a target condensate, or a component thereof. In some embodiments, the interaction is the manner in which the compound, or the portion thereof, and the condensate, or the component thereof, affect one another as evaluated in the dense phase and/or the light phase. In some embodiments, the interaction includes an aspect of the partition characteristic of the compound, or the portion thereof, for the condensate—which encompasses various disease-associated factors associated with the condensate partitioning of the compound, or the portion thereof. In some embodiments, the interaction includes an aspect of the partition characteristic of the component of the condensate for the condensate in the presence of the compound, or the portion thereof—which encompasses various disease-associated factors associated with the impact that the compound, or the portion thereof, has on the phase behavior of the condensate. In some embodiments, the compound is a test compound. In some embodiments, the compound is a reference compound. In some embodiments, the condensate is a target condensate. In some embodiments, the condensate is a reference condensate.

In other aspects, provided herein is a method of identifying a molecular target for a therapeutic drug useful for treating a disease, the method comprising identifying a condensate of interest using any one of the methods described herein; and identifying the molecular target based on an association and/or interaction with the condensate of interest. In some embodiments, the molecular target partitions in the condensate of interest. In some embodiments, the molecular target interacts with a component that associates with and/or partitions in a condensate of interest.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXEMPLARY EMBODIMENTS

Embodiment 1. A method of identifying a condensate of interest associated with a disease, the method comprising: (a) comparing a first condensate phenotype from a first cell model with a second condensate phenotype from a second cell model, wherein a difference between the first cell model and the second cell model is attributable to one or more disease-associated factors, and wherein at least one of the one or more disease-associated factors is introduced to either the first cell model or the second cell model; and (b) identifying the condensate of interest as associated with the disease based on a difference between the first condensate phenotype and the second condensate phenotype for the condensate of interest.

Embodiment 2. The method of embodiment 1, wherein the first condensate phenotype and the second condensate phenotype are each characterized by one or more phenotypic identifiers.

Embodiment 3. The method of embodiment 2, wherein the one or more phenotypic identifiers comprise an identifier selected from the group consisting of a condensate presence, absence, level, morphological feature, location, behavior, composition, and material property.

Embodiment 4. The method of any one of embodiments 1-3, wherein the one or more disease-associated factors associated with the disease comprise a factor selected from the group consisting of a genetic variant, post-translational modification variant, exogenous genetic material, presence of an endogenous compound, presence of an exogenous compound, a physical process, and environmental stimulus.

Embodiment 5. The method of any one of embodiments 1-4, wherein the second cell model is treated and/or engineered based on the one or more disease-associated factors associated with the disease.

Embodiment 6. The method of any one of embodiments 1-5, wherein the first cell model is treated and/or engineered based on the one or more disease-associated factors associated with the disease.

Embodiment 7. The method of any one of embodiments 1-6, further comprising obtaining the second cell model.

Embodiment 8. The method of any one of embodiments 1-7, further comprising producing the second cell model.

Embodiment 9. The method of any one of embodiments 1-8, further comprising obtaining the first cell model.

Embodiment 10. The method of any one of embodiments 1-9, further comprising producing the first cell model.

Embodiment 11. The method of any one of embodiments 1-10, further comprising obtaining the first condensate phenotype.

Embodiment 12. The method of embodiment 11, wherein obtaining the first condensate phenotype comprises measuring an association of a first marker with the condensate of interest.

Embodiment 13. The method of embodiment 12, wherein the first marker is a biological marker.

Embodiment 14. The method of embodiment 12 or 13, wherein the association of the first marker with the condensate of interest is determined using an imaging technique.

Embodiment 15. The method of embodiment 14, wherein the imaging technique comprises labeling the first marker.

Embodiment 16. The method of any one of embodiments 1-15, further comprising obtaining the second condensate phenotype.

Embodiment 17. The method of embodiment 16, wherein obtaining the second condensate phenotype comprises measuring an association of a second marker with the condensate of interest.

Embodiment 18. The method of embodiment 17, wherein the second marker is a biological marker.

Embodiment 19. The method of embodiment 17 or 18, wherein the association of the second marker with the condensate of interest is determined using an imaging technique.

Embodiment 20. The method of embodiment 19, wherein the imaging technique comprises labeling the second marker.

Embodiment 21. The method of any one of embodiments 1-20, further comprising determining the difference between the first condensate phenotype and the second condensate phenotype.

Embodiment 22. The method of embodiment 21, wherein determining the difference between the first condensate phenotype and the second condensate phenotype comprises a qualitative assessment.

Embodiment 23. The method of embodiment 21 or 22, wherein determining the difference between the first condensate phenotype and the second condensate phenotype comprises a quantitative assessment.

Embodiment 24. The method of any one of embodiments 21-23, wherein determining the difference between the first condensate phenotype and the second condensate phenotype comprises an in silico technique.

Embodiment 25. The method of any one of embodiments 1-24, wherein the condensate of interest is present in, or derived from, the first cell model.

Embodiment 26. The method of any one of embodiments 1-24, wherein the condensate of interest is absent in, or not derived from, the first cell model.

Embodiment 27. The method of any one of embodiments 1-25, wherein the condensate of interest is absent in, or not derived from, the second cell model.

Embodiment 28. The method of any one of embodiments 1-26, wherein the condensate of interest is present in, or derived from, the second cell model.

Embodiment 29. The method of any one of embodiments 1-28, wherein the condensate of interest belongs to a condensate type selected from the group consisting of a stress granule, cleavage body, p-granule, histone locus body, multivesicular body, neuronal RNA granule, nuclear gem, nuclear pore, nuclear speckle, nuclear stress body, nucleolus, Oct1/PTF/transcription (OPT) domain, paraspeckle, perinucleolar compartment, PML, nuclear body, PML, oncogenic domain, polycomb body, processing body, Sam68 nuclear body, and splicing speckle.

Embodiment 30. The method of any one of embodiments 1-29, wherein the disease is a monogenic disease.

Embodiment 31. The method of any one of embodiments 1-29, wherein the disease is a polygenic disease.

Embodiment 32. The method of any one of embodiments 1-31, wherein the disease is a multifactorial disease.

Embodiment 33. The method of any one of claims 1-32, wherein the disease is caused, at least in part, by a stimulus and/or an exogenous agent.

Embodiment 34. The method of embodiment 33, wherein the disease is caused by an infectious agent.

Embodiment 35. The method of any one of embodiments 17-34, wherein the first marker and the second marker are the same.

Embodiment 36. The method of any one of embodiments 17-34, wherein the first marker and the second marker are different.

Embodiment 37. A method of identifying a marker useful for identifying a condensate of interest associated with a disease, the method comprising: (a) assessing a plurality of candidate markers, or precursors thereof, for a level of association with one or more disease-associated factors of the disease; (b) assessing at least one of the plurality of candidate markers for a condensate affinity factor; and (c) identifying the marker from the plurality of candidate markers based on the marker having a desired level of association with the one or more disease-associated factors of the disease and having a desired condensate affinity factor.

Embodiment 38. The method of embodiment 37, further comprising identifying the one or more disease-associated factors of the disease.

Embodiment 39. The method of embodiment 37 or 38, wherein each of the one or more disease-associated factors of the disease is selected from the group consisting of a genetic variant, post translational modification variant, exogenous genetic material, presence of an endogenous compound, presence of an exogenous compound, a physical process, and environmental stimulus.

Embodiment 40. The method of any one of embodiments 37-39, further comprising identifying a gene associated with the one or more disease-associated factors of the disease.

Embodiment 41. The method of embodiment 40, further comprising identifying a gene expression product based on the identified gene, wherein the gene expression product, or a portion thereof, is used to populate the plurality of candidate markers, or precursors thereof.

Embodiment 42. The method of any one of embodiments 37-41, wherein the level of association of each candidate marker with the one or more disease-associated factors of the disease is based on a disease-causal factor score.

Embodiment 43. The method of embodiment 42, wherein the disease-causal factor score reflects the strength of association of each candidate marker with the one or more disease-associated factors of the disease.

Embodiment 44. The method of embodiment 42 or 43, further comprising assigning each candidate marker with the disease-causal factor score.

Embodiment 45. The method of any one of embodiments 37-44, wherein the condensate affinity factor is based on a condensate-association score.

Embodiment 46. The method of embodiment 45, wherein the condensate-association score reflects the strength of association of the candidate marker, or a portion thereof, with any condensate, a specific condensate, and/or a macromolecule associated with a condensate.

Embodiment 47. The method of embodiment 45 or 46, further comprising assigning the candidate marker with the condensate-association score.

Embodiment 48. The method of any one of embodiments 37-47, wherein identifying the marker from the plurality of candidate markers comprises a cumulative score based on the desired level of association with the one or more disease-associated factors of the disease and the desired condensate affinity factor.

Embodiment 49. The method of any one of embodiments 37-48, wherein the marker is a biological marker.

Embodiment 50. The method of any one of embodiments 37-49, wherein the marker is identified in silico.

Embodiment 51. The method of any one of embodiments 37-50, further comprising verifying the marker as useful for identifying a condensate of interest associated with the disease.

Embodiment 52. A method of identifying a condensate of interest associated with a disease, the method comprising: (a) comparing a first condensate phenotype from a first cell model with a second condensate phenotype from a second cell model, wherein the first condensate phenotype and the second condensate phenotype are obtained using a marker identified using the method of any one of embodiments 37-51, wherein a difference between the first cell model and the second cell model is attributable to one or more disease-associated factors of the disease, and wherein at least one of the one or more disease-associated factors is introduced to either the first cell model or the second cell model; and (b) identifying the condensate of interest as associated with the disease based on a difference between the first condensate phenotype and the second condensate phenotype for the condensate of interest.

Embodiment 53. A method of identifying a compound that modulates a condensate phenotype, the method comprising: (a) admixing the compound and a composition comprising a cell model; and (b) obtaining a resulting condensate phenotype of the composition, wherein a difference between the resulting condensate phenotype and a reference condensate phenotype identifies the compound as modulating the condensate phenotype.

Embodiment 54. A method of identifying a compound useful for treating a disease, the method comprising: (a) admixing the compound and a composition comprising a first cell model; (b) obtaining a resulting condensate phenotype of the composition, wherein the compound is identified as useful for treating the disease when the resulting condensate phenotype has a desired modulation of a phenotypic identifier associated with one or more disease-associated factors of the disease.

### EXAMPLES

### Example 1

This example demonstrates a method of identifying a condensate of interest associated with a disease.

Certain RBM20 mutants were identified as disease-associated factors associated with familial dilated cardiomyopathy (DCM). H9C2 cells were obtained as a cardiomyocyte parent cell model. Four derivative cell models were produced by engineering the H9C2 cells to express one of the following RBM20 markers (i) a wild type RBM20 polypeptide linked to a Dendra2 label, (ii) a R636S mutant RBM20 polypeptide linked to a Dendra2 label, (iii) a R636C mutant RBM20 polypeptide linked to a Dendra2 label, and (iv) a R636H mutant RBM20 polypeptide linked to a Dendra2 label. Following transfection, cells were incubated to allow for RBM20 polypeptide expression. Fluorescent images of the four cell models were captured using a DeltaVision wide-field deconvoluted system having a 60× oil objective.

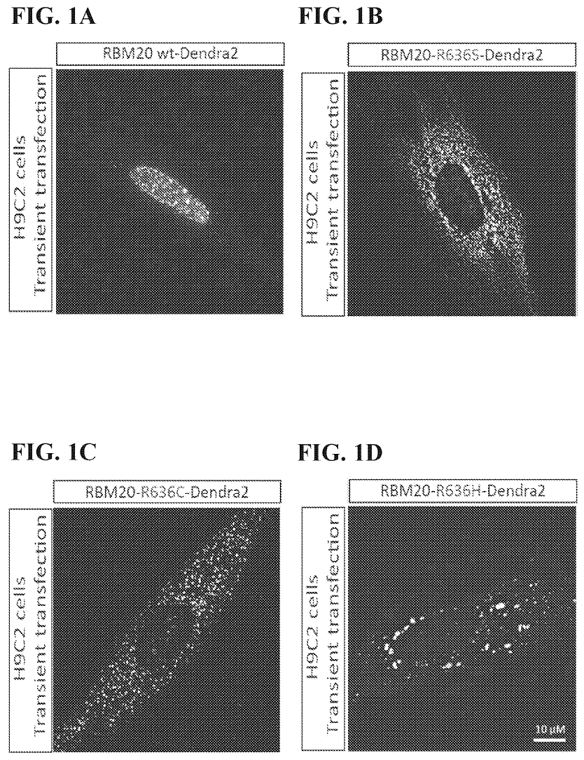

From the fluorescent images (FIG. 1A-1D), a condensate phenotype was obtained for each of the four RBM20 expressing cell models. The condensate phenotypes comprised condensate location, number, size, and shape. A comparison between the condensate phenotypes obtained from the cell models was then conducted. For example, as illustrated in FIGS. 1A-1D, in H9C2 cells transfected with a wild type RBM20 polypeptide, RBM20 condensates were primarily observed in the nucleus (FIG. 1A), and in H9C2 cells transfected with a RBM20 mutant polypeptide, RBM20 condensates were exclusively observed in the cytoplasm (FIG. 1B, R636S RBM20; FIG. 1C, R636C RBM20; FIG. 1D, R636H RBM20). Accordingly, the RBM20 condensates were identified as a condensate of interest associated with familial DCM.

### Example 2

This example demonstrates the large-scale analysis of genomic information to produce interaction maps useful for identifying cellular components associated with healthy and disease states. Such interactions may include both direct (e.g., physical contact between the genes) or indirect relationships (e.g., one gene turns on the production of other genes). Subsequently, the interaction maps may be used to identify disease-associated factors, identify markers (such as biological markers) useful for identifying a condensate phenotype and/or condensate of interest, identify (and/or engineer) relevant tissue and/or cell types, such as relevant to a disease and/or in which a drug screening assay will be performed, and identify a pathway in which a gene (such as a gene associated with a condensate) and/or a condensate of interest are functionally involved.

In brief, human genetics is used as a basis for evaluating and marking important regions of the genome associated with a disease. Such information is then combined with gene interaction information to create maps of gene interactions that modulate each disease. Next, for each set of genes, associated phenotypes (such as disease phenotypes) are used to break the mapped interactions into smaller groups, thereby adding detail to the map in terms of specific processes and cellular components that are associated with the disease. Additionally, loss-of-function genetics and animal knockout data is used to evaluate and screen the mapped interactions to increase the confidence of the findings.

More specifically, genotype-phenotype association summary statistics are obtained from a database, or re-derived as needed from case-control genotypes via logistic regression for a number of traits. Confounding variables in the regression model will be controlled for in the regression process. Next, the DAPPLE algorithm is used to generate protein interaction maps for each trait. In brief, the DAPPLE algorithm uses a greedy selection method to identify the largest protein-protein interaction (PPI) subnetwork that encompasses the most genes within defined genomic windows of peak loci for a trait. Subsequently, the algorithm repeats the process using the same loci now on scrambled PPI networks that preserve the degree for each gene node, thereby generating a background null distribution of PPI subnetworks for computing a statistical significance value for the originally discovered subnetwork. For each trait, a set of endophenotypes is collected which share heritability with the trait, and colocalization between the trait and its endophenotypes is computed to assign genes to endophenotypic classes in order to cluster the genes. The clusters are used to further break the interaction maps into modules. The findings above are validated using information regarding exome loss-of-function variants and publicly available mouse knockout data to validate findings.

The findings will provide any of disease-associated factors (such as useful for identifying and/or engineering a cell model), markers useful for identifying a condensate phenotype and/or condensate of interest, guidance on relevant tissue and/or cell types, such as relevant to a disease and/or in which a drug screening assay will be performed, and information regarding a pathway in which a gene (such as a gene associated with a condensate) and/or a condensate of interest are functionally involved.

### Example 3

This example demonstrates use of a workflow taught herein to identify and prioritize genes or non-coding variants predicted to be associated with a condensate and involved in the pathogenesis of Type 2 diabetes (T2D). Specifically, as described in more detail below, the method comprises assessing a plurality of candidate genes for a level of association with one or more disease-associated factors of T2D, and assessing a subset of candidate genes for plurality of condensate affinity factors. Identifying and prioritizing genes comprises use of a cumulative score as discussed below.

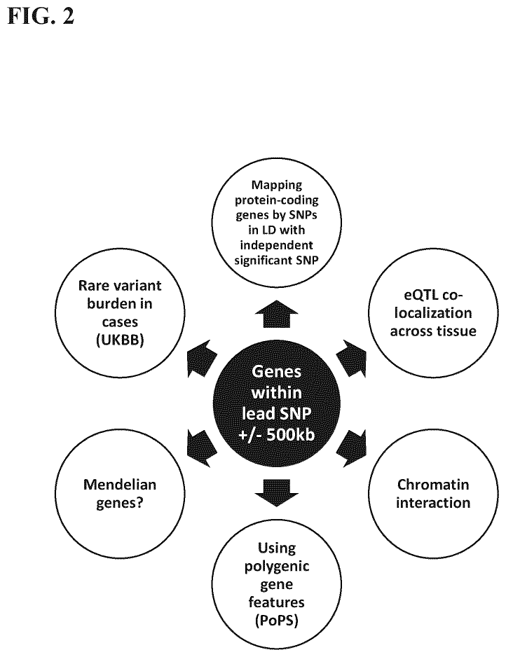

FIG. 2 shows an exemplary workflow of a method described herein. Data from genome-wide association studies (GWAS) was used to identify loci and genes associated with T2D. The human genome is composed of at least 20,000 known genes. Following the GWAS analysis a locus-to-gene table was produced listing 226 loci encompassing 2,540 genes (within ±500 kb of a lead SNP) with an association to T2D (a threshold of p<5×10⁻⁸ was used for this analysis). Our GWAS identified loci number was comparable to those identified in the literature (A. Mahajan et al., Nat Genet. 2018; 50(11):1505-1513; M. Vujkovic et al., Nat Genet. 2020; 52(7):680-691).

Prior to further analysis according to the methods described herein, Multi-marker Analysis of GenoMic Annotation (MAGMA) analysis of the GWAS data was performed to assess the members of the locus-to-gene table. Significantly enriched MAGMA gene sets that were Bonferroni corrected showed that top significantly enriched genes identified by the GWAS analysis were associated with relevant T2D biological pathways, molecular function, and cellular components (based on the Molecular Signatures Database (MsigDB)). However, no significantly enriched tissue types or condensate associations based on having more than one gene (or gene product) in a known condensate were identified from the MAGMA gene sets. This result illustrates the need for further condensate specific analysis, such as those described herein, to identify and prioritize genes associated with a condensate and involved in the pathogenesis of T2D.

Using the GWAS data, candidate genes/markers (including causal genes/variants (which causes the gene-disease association), target genes (which are affected by the causal variants), and other candidates (such as unknown candidate markers associated with T2D)) associated with the identified loci were evaluated to identify and prioritize genes of interest based on a weighted gene prioritization score. Using the 226 GWAS loci associated with T2D, the 2540 mapped genes within 500 kilobases of a lead SNP were independently assessed based on each of the following: (a) condensate-focused polygenic gene features (e.g., modified PoPS), (b) Mendelian gene analysis, (c) rare variant burden, (d) mapping protein-coding genes by SNPs in Linkage Disequilibrium (LD) with independent significant SNP, (e) eQTL colocalization across tissue, and (f) chromatin interaction.

Polygenic Priority Score (PoPS, Weeks et al., medRxiv, 2020), a gene prioritization method that leverages polygenic signals from GWAS and biological databases from various sources, was modified to include condensate-focused polygenic gene features. For PoPS, the data input included GWAS summary statistics and a gene membership input matrix to incorporate biological information from relevant datasets of gene pathways, protein-protein interactions, and single cell RNA-seq. In addition to gene pathway, protein-protein interaction, and single cell RNA-seq datasets, condensate membership information from a proprietary database and additional disease relevant gene expression data were included in the gene membership input matrix for the modified PoPS analysis. Of the 2540 mapped genes from the GWAS analysis, 1232 genes with high modified PoPS scores were identified.

Mendelian gene analysis was conducted using Online Mendelian Inheritance in Man (OMIM) compendium that links human genes/genotypes and genetic phenotypes, such as disease phenotypes. These phenotypes included (1) single-gene Mendelian disorders and traits, (2) susceptibilities to cancer and complex diseases, (3) variations that lead to abnormal but benign laboratory test values and blood groups, and (4) selected somatic cell genetic diseases. Of the 2540 mapped genes from the GWAS analysis, 580 genes had an OMIM phenotype and, following manual curation, 694 OMIM phenotypes were identified. Of these, 307 genes were associated with T2D relevant genetic disorders.

Rare variants converged in GWAS identified genes were assessed using databases, such as the UK BioBank (UKBB), which associates variants with phenotypes.

Protein-coding variant mapping was assessed by locating all SNPs in linkage disequilibrium with independent significant SNPs. From these SNPs, exonic and splicing SNPS were kept and mapped to corresponding protein-coding genes. Of the 2540 mapped genes from the GWAS data, 116 genes were identified to contain protein-coding variants.

Regulatory variant mapping was performed via eQTL colocalization. eQTL colocalization was performed to assess if a single variant was responsible for both GWAS and eQTL signals in a locus (a locus that explains a fraction of the genetic variance of a gene expression phenotype). A higher weight was given to genes with signals in disease relevant tissues. Of the 2,540 genes identified via the GWAS analysis, 193 genes were identified as having eQTL colocalization across tissues.

Regulatory variant mapping was also performed by chromatin interaction mapping (e.g., based on Hi-C or 3C data). A higher weight was given to genes with signals in disease relevant tissues. Of the 2,540 genes identified via the GWAS analysis, 1,038 genes were identified as associated with chromatin interactions.

Gene scoring and prioritization was performed using a weighted gene prioritization score including data from each of: (a) condensate-focused polygenic gene features (PoPS), (b) Mendelian gene analysis, (c) rare variant burden, (d) mapping protein-coding genes by SNPs in LD with independent significant SNP, (e) eQTL colocalization across tissue, and (f) chromatin interaction. Furthermore, it was required that each locus having 0-2 causal genes (e.g., based on literature, or based on fine-mapping of GWAS signals (see, e.g., H. Huang et al. Nature. 2017; 547(7662):173-178)) and a minimum weighted gene prioritization score. This resulted in the identification and prioritization of 228 genes from the 226 GWAS loci. Among these, 169 genes were with high confidence (i.e., with weighted gene prioritization score above a pre-determined threshold).

The 228 prioritized genes were then analyzed for condensate features, which contribute to the condensate-association score, including a probability of phase-separation formation (e.g., DeepPhase score; see, C. Yu et al., “Proteome-scale analysis of phase-separated proteins in immunofluorescence images,” Brief Bioinform. 2021 May; 22(3):bbaa187), predicted condensate formation (e.g., Pscore), presence of an intrinsically disordered region (IDR region) or a fraction of an IDR region, known association with a condensate, and protein image from the Human Protein Atlas (HPA). Further gene features were associated with each of the prioritized 228 genes, such as gene description, Uniprot ID and protein class, association with biological pathway and molecular function, related disease, cell-type specific RNA expression, and cellular location and secretome location.

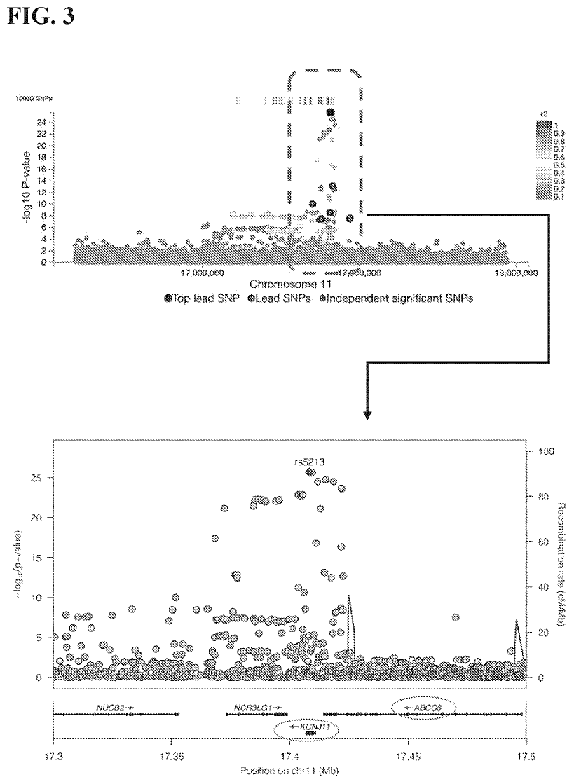

Based on the output from the above-described methodology, high accuracy of gene identification was achieved (e.g., match with gold standard T2D genes). Among which, KCNJ11 and ABCC8 were prioritized as genes of interest (see FIG. 3). KCNJ11 and ABCC8 are members of ATP-sensitive potassium channels and have a known association with T2D susceptibility, many patients with KCNJ11 or ABCC8 mutations can be successfully treated for years with sulfonylurea medications. KCNJ11 or ABCC8 thus serve as positive controls of marker or disease-associated factor of our study.

### Example 4

This example demonstrates a method of identifying a condensate of interest associated with a disease via a condensate phenotype.

Certain Desmoplakin (DSP), Desmoglein-2 (DSG2), and alpha-protein kinase 3 (ALPK3) mutants were identified to be associated with familial DCM. DSP and DSG2 are both crucial components for desmosome structures in cardiac muscle and epidermal cells, which function to maintain the structural integrity at adjacent cell contacts. ALPK3 is involved in cardiomyocyte differentiation.

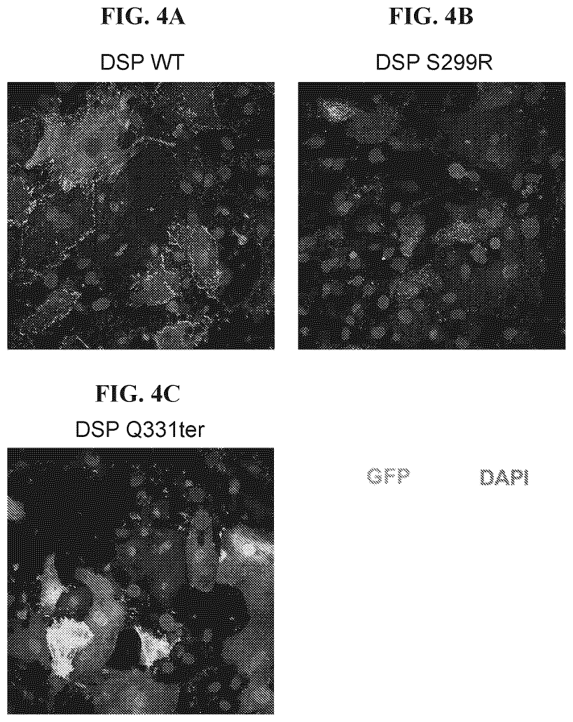

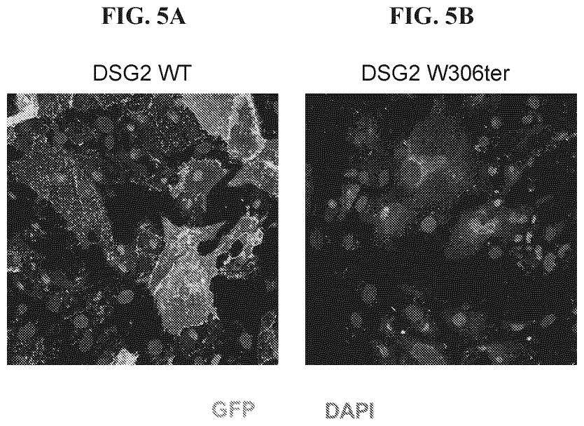

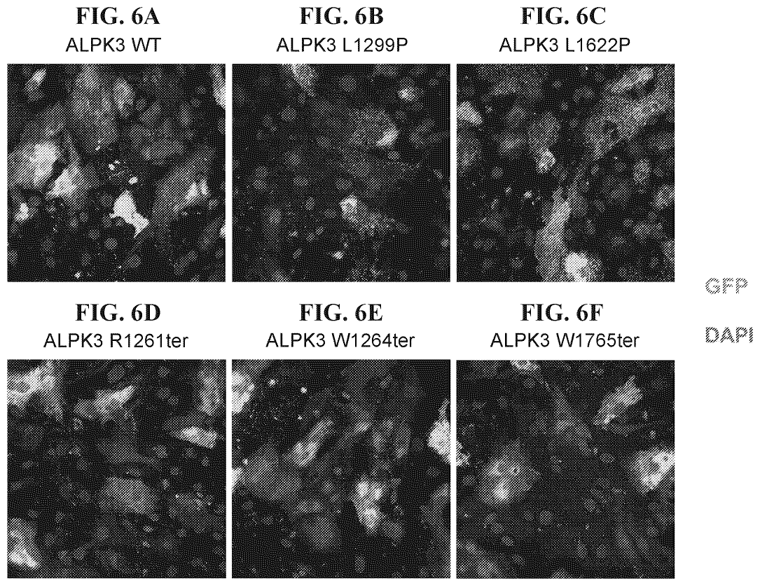

iCell® Cardiomyocytes, which were derived from induced pluripotent stem cells (iPSCs), were obtained as a cell model. 12 derivative cell models were produced by transfecting iCell® Cardiomyocytes with plasmids to express 1) GFP-labeled DSP wild type (WT) polypeptide “DSP-GFP-WT” (FIG. 4A); 2) GFP-labeled DSP S299R point mutation polypeptide “DSP-GFP-5299R” (FIG. 4B); 3) GFP-labeled DSP termination mutation polypeptide “DSP-GFP-Q331ter” (FIG. 4C); 4) GFP-labeled DSG2 wild type (WT) polypeptide “DSG2-GFP-WT” (FIG. 5A); 5) GFP-labeled DSG2 termination mutation polypeptide “DSG2-GFP-W306ter” (FIG. 513); 6) GFP-labeled ALPK3 wild type (WT) polypeptide “ALPK3-GFP-WT” (FIG. 6A); 7) GFP-labeled ALPK3 L1299P point mutation polypeptide “ALPK3-GFP-L1299P” (FIG. 6B); 8) GFP-labeled ALPK3 L1622P point mutation polypeptide “ALPK3-GFP-L1622P” (FIG. 6C); 9) GFP-labeled ALPK3 termination mutation polypeptide “ALPK3-GFP-R1261ter” (FIG. 6D); 10) GFP-labeled ALPK3 termination mutation polypeptide “ALPK3-GFP-W1264ter” (FIG. 6E); and 11) GFP-labeled ALPK3 termination mutation polypeptide “ALPK3-GFP-W1765ter” (FIG. 6F). Following transfection, cells were incubated for 48 hours to allow for polypeptide expression via the above-mentioned plasmids. Cells were stained with DAPI, a blue-fluorescent DNA stain. Fluorescent images of the 12 cell models were captured using a spinning disk confocal with 40× water objective using channels for GFP and DAPI.

From the fluorescent images, a condensate phenotype was obtained for each of the above-mentioned 12 cell models. The condensate phenotypes comprised condensate location, number, size, and/or shape. A comparison between the condensate phenotypes obtained from the cell models expressing wild type and variant polypeptides was then conducted, which showed changes in condensate phenotypes.

As illustrated in FIG. 4A, in iCell® Cardiomyocytes expressing wild type DSP, wild type DSP localized to puncta (DSP condensates) around the cell periphery. In cells expressing DSP S299R point mutation polypeptide, DSP puncta localized to the interior of the cells (FIG. 4B). In cells expressing DSP-GFP-Q331ter (FIG. 4C), DSP puncta were ablated and GFP levels were diffuse around the entire cell.

Similar to wild type DSP condensates, in iCell® Cardiomyocytes expressing wild type DSG2, wild type DSG2 localized to puncta (DSG2 condensates) around the cell periphery (FIG. 5A). In cells expressing DSG2 termination mutation polypeptide, DSG2 puncta were ablated and GFP levels were diffuse around the cell nucleus (FIG. 5B).

As illustrated in FIG. 6A, in iCell® Cardiomyocytes expressing wild type ALPK3, wild type ALPK3 localized to puncta (ALPK3 condensates) throughout the cytosol. In cells expressing ALPK3 L1299P (FIG. 6B) or L1622P (FIG. 6C) point mutation polypeptide, smaller and more numerous ALPK3 puncta were observed. In cells expressing ALPK3 termination mutation polypeptides, ALPK3 puncta were ablated and GFP levels were diffuse around the entire cell (FIGS. 6D-6F).

Accordingly, DSP, DSG2, and ALPK3 condensates were identified as condensates of interest associated with familial DCM. DSP, DSG2, and ALPK3 can serve as markers useful for identifying a condensate of interest associated with familial DCM.

What is claimed is:

1. A method of identifying a condensate of interest associated with a disease, the method comprising:
(a) comparing a first condensate phenotype from a first cell model with a second condensate phenotype from a second cell model,
wherein a difference between the first cell model and the second cell model is attributable to one or more disease-associated factors, and
wherein at least one of the one or more disease-associated factors is introduced to either the first cell model or the second cell model; and
(b) identifying the condensate of interest as associated with the disease based on a difference between the first condensate phenotype and the second condensate phenotype for the condensate of interest.

2. The method of claim 1, wherein the first condensate phenotype and the second condensate phenotype are each characterized by one or more phenotypic identifiers.

3. The method of claim 2, wherein the one or more phenotypic identifiers comprise an identifier selected from the group consisting of a condensate presence, absence, level, morphological feature, location, behavior, composition, and material property.

4. The method of any one of claims 1-3, wherein the one or more disease-associated factors associated with the disease comprise a factor selected from the group consisting of a genetic variant, post-translational modification variant, exogenous genetic material, presence of an endogenous compound, presence of an exogenous compound, a physical process, and environmental stimulus.

5. The method of any one of claims 1-4, wherein the second cell model is treated and/or engineered based on the one or more disease-associated factors associated with the disease.

6. The method of any one of claims 1-5, wherein the first cell model is treated and/or engineered based on the one or more disease-associated factors associated with the disease.

7. The method of any one of claims 1-6, further comprising obtaining the second cell model.

8. The method of any one of claims 1-7, further comprising producing the second cell model.

9. The method of any one of claims 1-8, further comprising obtaining the first cell model.

10. The method of any one of claims 1-9, further comprising producing the first cell model.

11. The method of any one of claims 1-10, further comprising obtaining the first condensate phenotype.

12. The method of claim 11, wherein obtaining the first condensate phenotype comprises measuring an association of a first marker with the condensate of interest.

13. The method of claim 12, wherein the first marker is a biological marker.

14. The method of claim 12 or 13, wherein the association of the first marker with the condensate of interest is determined using an imaging technique.

15. The method of claim 14, wherein the imaging technique comprises labeling the first marker.

16. The method of any one of claims 1-15, further comprising obtaining the second condensate phenotype.

17. The method of claim 16, wherein obtaining the second condensate phenotype comprises measuring an association of a second marker with the condensate of interest.

18. The method of claim 17, wherein the second marker is a biological marker.

19. The method of claim 17 or 18, wherein the association of the second marker with the condensate of interest is determined using an imaging technique.

20. The method of claim 19, wherein the imaging technique comprises labeling the second marker.

21. The method of any one of claims 1-20, further comprising determining the difference between the first condensate phenotype and the second condensate phenotype.

22. The method of claim 21, wherein determining the difference between the first condensate phenotype and the second condensate phenotype comprises a qualitative assessment.

23. The method of claim 21 or 22, wherein determining the difference between the first condensate phenotype and the second condensate phenotype comprises a quantitative assessment.

24. The method of any one of claims 21-23, wherein determining the difference between the first condensate phenotype and the second condensate phenotype comprises an in silico technique.

25. The method of any one of claims 1-24, wherein the condensate of interest is present in, or derived from, the first cell model.

26. The method of any one of claims 1-24, wherein the condensate of interest is absent in, or not derived from, the first cell model.

27. The method of any one of claims 1-25, wherein the condensate of interest is absent in, or not derived from, the second cell model.

28. The method of any one of claims 1-26, wherein the condensate of interest is present in, or derived from, the second cell model.

29. The method of any one of claims 1-28, wherein the condensate of interest belongs to a condensate type selected from the group consisting of a stress granule, cleavage body, p-granule, histone locus body, multivesicular body, neuronal RNA granule, nuclear gem, nuclear pore, nuclear speckle, nuclear stress body, nucleolus, Oct1/PTF/transcription (OPT) domain, paraspeckle, perinucleolar compartment, PML, nuclear body, PML, oncogenic domain, polycomb body, processing body, Sam68 nuclear body, and splicing speckle.

30. The method of any one of claims 1-29, wherein the disease is a monogenic disease.

31. The method of any one of claims 1-29, wherein the disease is a polygenic disease.

32. The method of any one of claims 1-31, wherein the disease is a multifactorial disease.

33. The method of any one of claims 1-32, wherein the disease is caused, at least in part, by a stimulus and/or an exogenous agent.

34. The method of claim 33, wherein the disease is caused by an infectious agent.

35. The method of any one of claims 17-34, wherein the first marker and the second marker are the same.

36. The method of any one of claims 17-34, wherein the first marker and the second marker are different.

37. A method of identifying a marker useful for identifying a condensate of interest associated with a disease, the method comprising:
(a) assessing a plurality of candidate markers, or precursors thereof, for a level of association with one or more disease-associated factors of the disease;
(b) assessing at least one of the plurality of candidate markers for a condensate affinity factor; and
(c) identifying the marker from the plurality of candidate markers based on the marker having a desired level of association with the one or more disease-associated factors of the disease and having a desired condensate affinity factor.

38. The method of claim 37, further comprising identifying the one or more disease-associated factors of the disease.

39. The method of claim 37 or 38, wherein each of the one or more disease-associated factors of the disease is selected from the group consisting of a genetic variant, post translational modification variant, exogenous genetic material, presence of an endogenous compound, presence of an exogenous compound, a physical process, and environmental stimulus.

40. The method of any one of claims 37-39, further comprising identifying a gene or a non-coding variant associated with the one or more disease-associated factors of the disease.

41. The method of any one of claims 37-40, wherein the level of association of each candidate marker with the one or more disease-associated factors of the disease is based on a disease-causal factor score.

42. The method of claim 41, wherein the disease-causal factor score reflects the strength of association of each candidate marker with the one or more disease-associated factors of the disease.

43. The method of claim 41 or 42, further comprising assigning each candidate marker with the disease-causal factor score.

44. The method of any one of claims 37-43, wherein the condensate affinity factor is based on a condensate-association score.

45. The method of claim 44, wherein the condensate-association score reflects the strength of association of the candidate marker, or a portion thereof, with any condensate, a specific condensate, and/or a macromolecule associated with a condensate.

46. The method of claim 44 or 45, further comprising assigning the candidate marker with the condensate-association score.

47. The method of any one of claims 44-46, wherein the condensate-association score is a composite score of a condensate function score and a condensate affinity score.

48. The method of claim 47, wherein the condensate function score is determined based on one or more factors of whether a genetic variation of the candidate marker or a portion thereof or the gene or the non-coding variant associated with the one or more disease-associated factors of the disease:
i) is within an intrinsically disordered region (IDR);
ii) is subject to a post-translational modification;
iii) affects splicing of the candidate marker or the gene associated with the one or more disease-associated factors of the disease;
iv) affects a chromatin state close to the gene or the non-coding variant associated with the one or more disease-associated factors of the disease; and
v) affects expression of the gene associated with the one or more disease-associated factors of the disease.

49. The method of claim 48, where the one or more factors each has a weight contributing to the condensate function score.

50. The method of claim 47, wherein the condensate affinity score is determined based on, in the candidate marker or a portion thereof or the gene associated with the one or more disease-associated factors of the disease, one or more factors of:
i) the presence, absence, amount, and/or degree of an IDR;
ii) the presence, absence, amount, and/or degree of a condensate-favoring motif; and
iii) the presence, absence, amount, and/or valency of an interacting domain.

51. The method of claim 50, where the one or more factors each has a weight contributing to the condensate affinity score.

52. The method of any one of claims 40-51, further comprising identifying a gene expression product based on the identified gene, wherein the gene expression product, or a portion thereof, is used to populate the plurality of candidate markers, or precursors thereof.

53. The method of any one of claims 37-52, wherein identifying the marker from the plurality of candidate markers comprises a cumulative score based on the desired level of association with the one or more disease-associated factors of the disease and the desired condensate affinity factor.

54. The method of any one of claims 37-53, wherein the marker is a biological marker.

55. The method of any one of claims 37-54, wherein the marker is identified in silico.

56. The method of any one of claims 37-55, further comprising verifying the marker as useful for identifying a condensate of interest associated with the disease.

57. A method of identifying a condensate of interest associated with a disease, the method comprising:
(a) comparing a first condensate phenotype from a first cell model with a second condensate phenotype from a second cell model,
wherein the first condensate phenotype and the second condensate phenotype are obtained using a marker identified using the method of any one of claims 37-56,
wherein a difference between the first cell model and the second cell model is attributable to one or more disease-associated factors of the disease, and
wherein at least one of the one or more disease-associated factors is introduced to either the first cell model or the second cell model; and
(b) identifying the condensate of interest as associated with the disease based on a difference between the first condensate phenotype and the second condensate phenotype for the condensate of interest.

58. A method of identifying a compound that modulates a condensate phenotype, the method comprising:
(a) admixing the compound and a composition comprising a cell model; and
(b) obtaining a resulting condensate phenotype of the composition,
wherein a difference between the resulting condensate phenotype and a reference condensate phenotype identifies the compound as modulating the condensate phenotype.

59. A method of identifying a compound useful for treating a disease, the method comprising:
(a) admixing the compound and a composition comprising a first cell model;
(b) obtaining a resulting condensate phenotype of the composition,
wherein the compound is identified as useful for treating the disease when the resulting condensate phenotype has a desired modulation of a phenotypic identifier associated with one or more disease-associated factors of the disease.
